(19) United States
(12) Patent Application Publication
(10) Pub. No.: US 2024/0165228 A1
(43) Pub. Date: May 23, 2024

(54) TARGETED INHIBITION OF DKK3 TO SENSITIZE TUMORS TO IMMUNOTHERAPY

(71) Applicant: Duke University, Durham, NC (US)

(72) Inventors: Zachary Hartman, Durham, NC (US); Herbert Lyerly, Durham, NC (US); Timothy Trotter, Durham, NC (US)

(21) Appl. No.: 18/513,095

(22) Filed: Nov. 17, 2023

Related U.S. Application Data
(60) Provisional application No. 63/522,602, filed on Jun. 22, 2023.
(60) Provisional application No. 63/426,132, filed on Nov. 17, 2022.

(51) Int. Cl. A61K 39/395; A61P 35/04

(52) U.S. Cl. CPC A61K 39/3955; A61P 35/04; A61K 2121/00

(57) ABSTRACT

Disclosed herein are methods of methods of inhibiting growth of a dormant cancer cell comprising administering an effective amount of a DKK3 inhibitor to a subject in need thereof to inhibit growth of the dormant cancer cell.

### CROSS-REFERENCE TO RELATED APPLICATIONS

The present application claims priority to U.S. Provisional Patent Application No. 63/426,132, filed Nov. 17, 2022, and U.S. Provisional Patent Application No. 63/522,602, filed Jun. 22, 2023, the entire contents of which are hereby incorporated by reference.

### STATEMENT REGARDING FEDERALLY SPONSORED RESEARCH

This invention was made with government support under T32CA009111 and R01CA238217-01A1/02S1 awarded by National Institutes of Health and W81XWH-20-1-0346 awarded by the Department of Defense. The government has certain rights in the invention.

### SEQUENCE LISTING

A Sequence Listing accompanies this application and is submitted as an ASCII text file of the sequence listing named “155554_00724_Sequence_Listing.xml” which is 1,815 bytes in size and was created on Nov. 14, 2023. The sequence listing is electronically submitted via EFS-Web with the application and is incorporated herein by reference in its entirety.

### BACKGROUND OF THE INVENTION

Therapies targeting early stages of breast cancer (BC) are increasingly effective and survival has steadily improved in the last two decades. Despite these successes, a significant portion of survivors (approximately 30%) will eventually experience locoregional or metastatic recurrence, even when there was no clinical evidence of disease after initial therapy. Among solid tumors, BC has a propensity for delayed relapse with distinct patterns of recurrence based upon molecular subtype. Those with triple negative breast cancer (TNBC), defined by lack of the estrogen receptor (ER), progesterone receptor (PR), and human epidermal growth factor receptor 2 (HER2), are particularly at risk of distant recurrence with a shorter window than other subtypes (33.9% vs 20.4%; 2.6 vs 5 years respectively). In contrast, ER+tumors can recur up to decades after treatment of the primary tumor and appear to have a consistent risk of recurrence over time. Regardless, the time between remission and relapse represents a critical window of opportunity to eliminate residual tumor cells before developing resistance mechanisms to form new, overt tumors that are exceedingly challenging to treat, leaving an unmet need to treat tumors possessing characteristics of this phenomenon of delayed relapse, often described generally as tumor dormancy.

The advent of immunotherapy highlighted the role that immune cells play in an evolving tumor, even in tumor dormancy. Organ transplants provided some of the earliest evidence that the immune system is crucial for preventing outgrowth of otherwise dormant tumor cells, when occult tumors from donated organs began growing in the context of the immunosuppressed recipient. Recently, direct evidence of T cell prevention of metastatic outgrowth was described in BC, although it remains unknown if T cells are capable of eliminating residual tumors in a quiescent/minimally proliferative state. If the hope of immunotherapy is to provide a lasting cure for patients, the mechanisms that prevent adaptive immunity from eliminating remaining tumor cells must be understood. Therefore, it was sought to uncover the mechanisms by which residual tumor cells resist immune-mediated elimination to eventually become an active tumor.

The present disclosure demonstrates that dormant tumors are not invisible to T cell recognition, but instead survive long-term even in the presence of activated tumor antigen-specific CD8+T cells. To survive, it was found that dormant tumors induce a CD4/Treg shifted T cell profile via secreted factors and identify the Wnt pathway antagonist, Dickkopf WNT Signaling Pathway Inhibitor 3 (DKK3), as a crucial mediator of this effect. Together, the data presented here provide insight into novel protective barriers that prevent elimination of dormant tumors and lay the foundation for new targets that may be combined with current immunotherapies to provide more durable responses for patients.

### BRIEF SUMMARY OF THE INVENTION

The present disclosure relates to method of inhibiting DKK3 as a means of inhibiting dormant cancer growth and/or CD4+T regulatory cells.

In a first aspect, the disclosure provides a method of inhibiting growth of a dormant cancer cell comprising administering an effective amount of a DKK3 inhibitor to a subject in need thereof to inhibit growth of the dormant cancer cell. In some aspects, the dormant cancer cell is a cell from a cancer selected from the group consisting of a breast cancer, a pancreatic cancer, a prostate cancer, a brain cancer, an ovarian cancer, a liver cancer, and a lung cancer. In some aspects, the dormant cancer cell is from a triple-negative breast cancer or an estrogen receptor positive breast cancer.

In some aspects the subject previously received a cancer therapy treatment for a cancer, and in some aspects the cancer therapy treatment for the cancer resulted in remission of the cancer.

In some aspects, the DKK3 inhibitor is an antibody or antibody fragment. In some aspects, administration results in an increase in Foxp3+T regulatory cells in the subject. In some aspects administration of the effective amount of the DKK3 inhibitor increases CD8+T cell proliferation and/or decreases CD4+T cell proliferation. In some aspects, the method further comprises administering an immunotherapy; in some aspects the immunotherapy comprises an immune checkpoint inhibitor, and in some aspects the immune checkpoint inhibitor is selected from the group consisting of a CTLA-4 antagonist, a PD-1 antagonist, a PD-L1 antagonist, an OX40 agonist, a LAG3 antagonist, a 4-1BB agonist, or a TIM3 antagonist. In some aspects, the method further comprises administering at least one additional cancer therapy; and in some aspects the at least one additional cancer therapy is selected from the group consisting of an anti-cancer vaccine, radiation, chemotherapy, surgery, immunotherapy, gene therapy, hormone therapy, anti-angiogenic therapy, and cytokine therapy.

In some aspects, the dormant cancer cell comprises at least one metastasis. In some aspects, the dormant cancer cell is part of at least one secondary tumor.

In a second aspect, the present disclosure provides a method of inhibiting CD4+Treg cells in a subject comprising administering an effective amount of a DKK3 inhibitor to a subject in need of inhibition of a CD4+T reg cell response, wherein subject has a decrease in Foxp3+T regulatory cells after administration of the DKK3 inhibitor. In some aspects, the DKK3 inhibitor is an antibody or antibody fragment, and in some aspects the administration of the effective amount of the DKK3 inhibitor increases CD8+T cell proliferation and/or decreases CD4+T cell proliferation.

In a third aspect, the present disclosure provides a method of inhibiting cancer recurrence in a subject previously treated for a cancer comprising administering an effective amount of a DKK3 inhibitor to the subject following completion of a cancer therapy in the subject.

In a fourth aspect, the present disclosure provides a method of treating a subject with cancer comprising: (a) obtaining a sample of cancer cells from the subject; (b) determining the level of expression of DKK3 in the cancer cells; (c) treating the cancer in the subject; (d) administering a DKK3 inhibitor to the subject with increased expression levels of DKK3 in the cancer cells in step (b) after completion of treatment of the subject for the cancer in step (c).

### BRIEF DESCRIPTION OF THE DRAWINGS

FIG. 1: The adaptive immune system does not impact tumor dormancy and long-term persistence in mammary D2.1 tumors. A Tumor growth in the mammary fat pad of 10⁶ parental D2A1 cells in Balb/c or SCID-beige mice (n=5/group). B D2.OR tumor growth of parental 10⁶ cells in Balb/c or SCID-beige mice (n=5/group). Comparisons were performed by two way ANOVA at time of first euthanasia (A,B). C Parental D2.1 growth of 10⁶ cells implanted into the mammary fat pad of Balb/c (n=5) or SCID-beige (n=8) mice. D Representative images (left) or quantification (right) of Ki67+eGFP+tumor cells after MFP implantation of 10⁶ D2.1-eGFP cells in Balb/c mice and collection after 4 or 12 weeks. Tumor sections were stained for nuclei (DAPI: pseudocolor gray), eGFP (green) or Ki67 (purple). Scale bars indicate 100 μm and statistical comparison by two-tailed t test. E Flow cytometry analysis of MHC-I surface expression on cultured D2A1, D2.OR, and D2.1 cells. F 10⁶ D2A1-eGFP, D2.OR-eGFP, or D2.1-eGFP single cell clones (n=5/group) were implanted into the mammary fat pad of female Balb/c mice. Tumors were resected at the indicated time point, digested, and cultured ex vivo for a pure tumor cell population. G Left panels: Representative flow plots for eGFP expression of ex vivo D2A1, D2.OR, or D2.1 tumors compared to parental (no eGFP) cells or respective single cell clones on the day of injection. Right panel: Quantification of the percent of cultured tumor cells that maintained eGFP expression after in vivo selection. Statistical comparisons were performed by one-way ANOVA with Tukey's post-hoc correction. Error bars represent mean±SEM.

FIG. 2: D2.1 tumor cells are enriched in mammary progenitor genes and maintain a hybrid E/M profile in vivo associated with immune dysfunction. A RNAseq was performed on D2A1 and D2.1 cells cultured in vitro. Shown are the top ten upregulated (black fill) and downregulated (white fill) Gene Ontology (GO) Biological Process (BP) pathways in D2.1 compared to D2A1. B Heatmap of selected significantly differentially expressed genes related to markers of mammary epithelium, antigen presentation via MHC I, a mesenchymal phenotype, and mammary epithelial cell differentiation is shown. C D2A1 and D2.1 tumors were implanted into the MFP (10⁶/mouse) and collected at approximately 500 mm³ for RNAseq (n=4/group). Shown are selected differentially expressed genes as in B. D GSEA of upregulated gene signatures in D2.1 tumors associated with mammary stem cells, luminal progenitor cells, and luminal mature cells compared to D2A1. E GSEA of upregulated gene sets in D2.1 tumors associated with late recurrence or F late distant metastasis in human breast cancer. G GO BP pathway analysis of D2.1 vs D2A1 tumors in Balb/c mice. 8 of the top 10 differentially expressed pathways were directly related to immune function. H CIBERSORT analysis of D2.1 and D2A1 Balb/c tumors. Statistical comparisons were performed by two-tailed t test and error bars represent mean±SEM. I GSEA of T cell anergy in D2.1 vs D2A1 Balb/c tumors.

FIG. 3: Dormant tumors and are highly infiltrated by T cells but induce a Treg-rich microenvironment. A 10⁶ D2A1 or D2.1 cells were implanted into the mammary fat pad of Balb/c mice. Small, volume-matched D2Al tumors (“Early D2A1”; n=6), large, duration-matched D2A1 (“D2A1”; n=4) tumors, or small D2.1 (n=4) tumors were collected as indicated. B Final tumor volume of tumors in A. C Flow cytometry analysis of total T cells (CD45+CD11b−CD4+or CD8β+) in total immune cells (CD45+) in tumors at time of euthanasia. D Percent of CD8 cells in total CD45+cells. E Percent of CD4 cells in total CD45+cells. F Left: Flow plots of CD4+Foxp3+cells in D2A1 and D2.1 tumors taken at day 35 (shown gated on CD45+cells). Right: Quantification of Tregs in tumors from A. G The CD8+cell to Treg (CD4+FoxP3+) ratio in total T cells by flow cytometry. One way ANOVA with Tukey's post-hoc analysis was used to compare 3 groups for B-G. H Representative IHC image of tumor border from endpoint D2A1 tumors. Dashed lines indicate stromal/tumor interface and arrows indicate positively stained CD8 (top) or CD4 (bottom) cells. I Representative images of sized-matched D2A1 or D2.1 tumor interiors stained for CD8 (top) or CD4 (bottom). J Quantification of CD8+or K CD4+cells/mm² in D2A1 and D2.1 tumor interiors (n=5 each). L Representative images (left) and quantification (right) of Foxp3+cells in tumors from H-K. Scale bars represent 50 μm (H, I, L). Density analysis was performed on 5 random fields/tumor using ImageJ software and comparisons were performed via two-tailed t test (J-L). M, N Correlation of endpoint volume and CD8+density (M) or FoxP3+density (N) in D2.1 tumors of different sizes (n=17). Pearson correlation is displayed in each plot. Error bars represent mean±SEM.

FIG. 4: Dormant D2.1 tumors evade antigen-specific killing by activated, tumor specific CD8+cells. A Representative flow plots (left) of PD-1 and CD39 expression on CD8+cells from endpoint D2A1 or D2.1 tumors and quantification (right) of PD-1+CD39+cells in total CD8+T cells in tumors or spleens of tumor-bearing Balb/c animals. Statistical comparisons were performed by two-tailed t test. B Jedi T cells express a TCR specific to eGFP₂₀₀₋₂₀₈ enabling antigen-specific targeting of eGFP+tumor cells. C In vitro killing of D2A1 cells plated with activated Jedi T cells at indicated ratio. D In vitro killing of D2.1 cells plated with activated Jedi T cells. Values were normalized to 0:1 E:T ratio for each condition and were compared by Šídák's two-way ANOVA. E Ex vivo expanded Jedi T cells or naïve splenocytes were transferred to naïve mice and 10⁶ D2A1/D2.1 eGFP expressing cells were implanted into the mammary fat pad after two days (n=5/group). F Growth plot of D2A1 or G D2.1 tumors respectively. P value displayed by Šídák's two-way ANOVA at end of experiment. Error bars represent mean±SEM.

FIG. 5: D2.1 cells directly induce CD4+Tregs which can be targeted in vivo. A Whole Jedi splenocytes were stained with CellTrace and stimulated with anti-CD28 antibodies+GFP₂₀₀₋₂₀₈ peptides in conditioned medium (CM) from D2A1 cells or D2.1 cells for 3 days followed by flow cytometric analysis. B Total T cells (CD45+CD11b−CD4+or CD8β+) in immune cells (CD45+) at the end of culture. C Representative flow plot of CD4 and CD8 T cells in CD45+cells and proportion of total CD4 and CD8 cells in CD45+cells. D, E Representative flow plot (left) and quantification of divisions (right) of CellTrace stained CD8+cells (D) or CD4+cells (E) in D2A1 or D2.1 CM. F Representative plot (left) and quantification (right) of FoxP3+Tregs in D2A1 and D2.1 CM after 3 days in culture. G CD8: Treg ratio in T cells in D2A1 or D2.1 CM. All conditions were performed in triplicate. Statistical comparisons were performed by two-tailed ttest (B, C, F, G) or Šídák's two-way ANOVA (D, E). H Tumor growth of D2.1 tumors (10⁶ cells/mammary fat pad) treated with anti CTLA4 antibodies (n=10) or isotype control (n=9) upon reaching 150 mm³. I D2.1 (10⁶ cells/mammary fat pad) were implanted into female FoxP3-DTR mice and received 0 ng/g DT (Untreated) or 5 doses of 25 ng/g every 4 days (arrows) beginning at day 35 (DT Early) or day 70 (DT late). J D2.1 tumor volume comparing all Untreated (n=14) mice to DT Early (n=9) at time of DT Early euthanasia (left) or comparing remaining Untreated mice to DT Late (n=8 each) at end of experiment (right). Statistical comparisons for H, J were performed by two-way ANOVA and P value shown is at the final timepoint. Error bars represent mean±SEM.

FIG. 6: D2.1-derived DKK3 is essential for tumor persistence and mediates T cell fate/function. A Differential expression analysis of D2.1 vs D2A1 tumors in Balb/c animals from FIG. 2. Dkk3 is highlighted with empty circle (log2 fold change=4.97; padjust=3.14×10⁻¹⁰⁸). B Normalized counts of Dkk3 from bulk RNAseq of D2.1 or D2A1 tumors implanted into Balb/c or SCID-beige animals. Counts were compared by one-way ANOVA with Holm-Šídák's multiple comparisons test. C Quantitative PCR of Dkk3 transcripts in samples from B. P values by one-way ANOVA with Holm-Sidak's multiple comparisons test. D Growth of 10⁶ D2.1 cells expressing shScramble (n=6) or shDKK3 (n=8) vectors in the MFP of Balb/c mice. E Growth of 10⁶ D2.1 control (n=10) or two independent DKK3 KO lines (n=5 each) in the MFP of Balb/c mice. Statistical comparisons are by two way ANOVA at end of experiment (D, E). F Growth of D2.1 control or DKK3 KO cells (n=5 each) in the MFP of SCID beige mice. G Bead-isolated Jedi CD8 cells were cultured in D2.1 shScramble or shDKK3 CM with antigen presenting cells (APCs) and eGFP₂₀₀₋₂₀₈ peptide. Representative flow plot (left) and quantification (right) of CD8 cell divisions in D2.1 CM are shown. H Jedi CD8 cells cultured as in G with the addition of equal numbers of bead-isolated CD4 cells. I Representative plot of CD4+and CD8+cells (gated on CD45+; left) and quantification (right) of CD4 cells in CD45+cells in D2.1 CM. J Representative plots (left) and quantification of Tregs in D2.1 CM. K CD8:Treg ratio after culture in D2.1 CM. L IFNγ ELISA after T cell culture in D2.1 CM. Statistical comparisons were performed by two-tailed ttest (I, J, K), one-way ANOVA with Tukey's correction (L), or Šídák's two-way ANOVA (G, H). Error bars represent mean±SEM.

FIG. 7: DKK3 is associated with poor survival, decreased effector function, and metastasis in human BC and promotes tumor growth in an immunocompetent setting. A Survival plots of human breast cancer stratified by high and low Dkk3 expression in the KMPlotter cohort. (Basal=477, Her2=348, Luminal A=903, Luminal B=676). B Survival plots of TCGA human breast cancers based upon Dkk3 expression. C Correlation analysis of Dkk1, Dkk2, and Dkk3 with immune-related genes in TCGA, KMPlotter, and METABRIC datasets. Individual p values are contained within Table 2. D Dkk3 expression in MBCProject samples with or without metastases present at time of sample collection. Statistical comparisons were performed by two-tailed t test. E Growth of parental, eGFP expressing only, or DKK3 eGFP D2.OR MFP tumors (10⁶ cells; n=5 each) in female Balb/c mice. Comparisons shown are by Tukey's two-way ANOVA at end of experiment. F Representative flow plot (left) and quantification (right) of Tregs in total CD4 cells in tumors from E. G Effector phenotype (CD44+CD62l−) of CD8 T cells in spleens of mice from E. Statistical analysis of F, G was performed by two-tailed t test. An outlier was identified for G (right; empty triangle) using the outlier analysis function in GraphPad Prism (ROUT method, q=1%) and the p value displayed excludes the outlier (p=0.1309 included). Error bars represent mean±SEM.

FIG. 8: A In vitro growth of D2A1 and D2.1 cells. Statistical comparison by two way ANOVA. B Phase contrast image of D2.1 cells at confluence. C Growth of 10⁶ D2A1 (n=3), 10⁶ D2.1 (n=3), or a mix of 5×10⁵ D2A1 and 5×10⁵ D2.1 cells (n=3) implanted into the mammary fat pad of female Balb/c mice. D Representative flow plot and MFI of D2A1 (n=4) or D2.1 (n=4) tumors after 35 days in mice. E PD-L1 surface expression on D2A1, D2.OR, and D2.1 cells in vitro (left) or D2A1 or D2.1 tumors from D. F CD47 expression of D2A1, D2.OR, and D2.1 cells in vitro. Comparisons were performed by two-tailed ttest (D,E). Error bars represent mean±SEM.

FIG. 9: A Reactome analysis of D2.1 or D2A1 cells related to FIG. 5A. Displayed are the top ten upregulated (black fill) and downregulated (white fill) pathways. B Quantitative PCR in parental D2A1, D2.OR, and D2.1 cells of select genes associated with FIG. 2B. Error bars represent mean±SD. P values were determined by one-way ANOVA with Tukey's post-hoc correction. C Esr1 and Greb1 gene expression from RNAseq of D2.1 or D2A1 cells. Error bars represent mean±SD. P values between D2.1 and D2A1 were determined by two-tailed ttest. D Representative flow plots of surface staining for CD24 and CD44 in cultured D2A1, D2.OR, and D2.1 cells. E Staining of CD104 and CD44 in cultured D2 cells. F Surface staining for CD104 and CD44 of D2.OR parental cells (left) or independent populations of surviving, residual D2.OR nodules recovered over 100 days after implantation into the MFP of Balb/c mice (right).

FIG. 10: A Growth of 10⁶ D2A1 or D2.1 cells implanted into the MFP of female Balb/c or SCID beige mice related to FIG. 2. Comparison shown is by two way ANOVA at time of collection for sequencing. B Correlation of RNA-seq data of D2.1 and D2A1 tumors from A. C Principal component analysis (PCA) of RNA-seq from tumors in A. D Ten most downregulated Reactome pathways in Balb/c D2.1 tumors compared to Balb/c D2A1. E Proliferation related GSEA enrichment plots in D2.1 vs D2A1 tumors in Balb/c (left) or SCID beige (right) mice. F Heatmap of differentially expressed dormancy-related genes in Balb/c D2.1 tumors compared to D2A1. G Venn diagram of tumors from A with annotated genes.

FIG. 11: A GSEA of Balb/c D2.1 vs D2A1 tumors related to Myc signaling. B Quantitative PCR analysis of Empty vector control or D2.1 cells engineered to overexpress MycT58A. Groups were compared by two-tailed t test and error bars represent mean±SD. C In vitro growth of cells from A. Error bars represent mean±SEM and comparisons were performed by Šídák two-way ANOVA. D Growth of 10⁶ D2.1 cells overexpressing MycT58A or Empty vector control cells in the MFP of female Balb/c of SCID beige mice. Comparisons shown are by two way ANOVA with Tukey's post hoc correction and error bars represent mean±SEM.

FIG. 12: A Representative gating strategy (from tumor) used to identify T cell populations. The same gating strategy was used for tumors and spleens in vivo as well as subsequent in vitro experiments. B Tumor growth plot associated with FIG. 3H-L. C Triple IHC for CD4, CD8, and FoxP3 in D2.1 tumor. Arrows indicate double labeled CD4+FoxP3+cells and scale bars represent 25 μm. D Overall tumor growth plots of tumors from 4A. Error bars represent mean±SEM.

FIG. 13: A Individual growth of tumors from FIG. 5H. B T cell profiles of tumors from A. An outlier was identified (right; empty square) using the outlier analysis function in GraphPad Prism (ROUT method, q=1%) and the P value displayed excludes the outlier (p=0.3173 included). C Representative flow plot (left) and individual values (right) of Tregs in spleens 2 days post final DT (25 ng/g) treatment in naïve animals. D, E Growth of individual tumors from FIG. 5J. F CD8 CD39+PD-1+cells in Untreated or DT late tumors from E. Statistical comparisons of two groups were performed by two-tailed ttest and error bars represent mean±SEM.

FIG. 14: A Quantitative PCR analysis of Dkk3 mRNA expression in parental D2A1, D2.OR, and D2.1 cells. B Analysis of Dkk3 mRNA in parental, shScramble control, or shDKK3 D2.1 cells by quantitative PCR. C ELISA for DKK3 protein on CM from D2.1 shScramble or shDKK3 cells. D Individual D2.1 tumor growth related to FIG. 6C. E Quantitative PCR analysis of Dkk3 mRNA expression after tumors from D were digested and plated to generate pure tumor populations ex vivo. Left panel is individual tumors and right panel shows the mean of each group. FELISA for DKK3 protein in CM from D2.1 non-targeting Crispr control cells or two independent guide RNAs directed towards Dkk3. G Growth of D2.1 cells from F. Comparisons of two groups were performed by two-tailed t test (C) and three groups were performed by one-way ANOVA with Tukey's post-hoc analysis (A, B, F). Error bars represent mean±SEM.

FIG. 15: A Representative flow plot (left) and quantification of CD8 cell divisions after 3 days when whole Jedi splenocytes were cultured in D2.1 Crispr control or Crispr DKK3 CM with anti-CD28 antibodies and eGFP₂₀₀₋₂₀₈ peptide. Statistical comparisons were performed using two-way ANOVA with Dunnett's multiple comparisons test. B Quantification of Tregs in CD4 cells from A. C Ratio of CD8 cells to Tregs in cultures from A. Comparisons for B, C were performed by one-way ANOVA with Tukey's post-hoc analysis. Error bars represent mean±SEM.

FIG. 16: A Dkk3 expression by ER positivity in TCGA BC samples. Comparisons were performed by two-tailed t test. B Single-cell RNA-sequencing of 26 human breast tumor samples accessed via the Broad Institute single-cell portal. Dkk3 expression is shown both overlaid on UMAP plot and as individual expression values grouped by annotated cell type. C Dkk3 expression overlaid on UMAP plot of cancer epithelial cells of ER+, HER2+, or TNBC after sub-clustering.

FIG. 17: A ELISA for DKK3 in CM from D2.OR cells engineered to express DKK3 or Empty control. B Growth of cells in culture from A. C Surface expression of CD44 and CD104 in D2.OR cells from A expressing DKK3 or Empty control. D Growth of 10⁶ D2.OR DKK3 or Empty control cells in the MFP of female Balb/c mice with p value at time of D2.OR DKK3 euthanasia by two way ANOVA shown. E ELISA on CM from D2.OR eGFP only or D2.OR eGFP DKK3 cells. F ELISA for DKK3 protein on CM (left) and growth in vitro (right) of D2A1 eGFP Empty control or eGFP DKK3 expressing cells. G Growth of control or D2A1 eGFP DKK3 tumors (10⁶ cells) in the MFP of female Balb/c or SCID beige mice with comparison by Šídák's two-way ANOVA at end of experiment shown. H Serum DKK3 levels by ELISA at time of euthanasia from G. I Effector CD8 T cells in tumors of mice from G. J T cell phenotyping in spleens of Balb/c mice from G. Effector cells were defined as CD44+CD62l−and Naïve cells were defined as CD44-CD62l+. K Anti-eGFP antibodies in serum of Balb/c mice from G normalized by tumor volume and compared by two-way ANOVA. Statistical analysis of two groups (A, E, F, H, I, J) was performed by two-tailed ttest. Error bars represent mean±SEM.

FIG. 18: D2.1 cells were implanted into the mammary fat pad of female Balb/c mice to determine the activity of DKK3 blockade in vivo. Beginning on day 3 mice (anti DKK3 Early) were treated bi-weekly with anti-DKK3 antibodies (clone 4.22; 450 μg/mouse; n=5) or isotype control (clone MOPC-21; 450 μg/mouse/; n=10). On day 65, half of the isotype control mice began bi-weekly treatment with anti-DKK3 antibody (anti DKK3 Late). Left panel: Tumor growth plot for the duration of the experiment. Right panel: Tumor growth plot during the latent phase demonstrating early effectiveness of anti-DKK3 blockade (1/5 tumors were rejected completely). Comparisons shown are by Sidak's 2-way ANOVA.

FIG. 19: A. Quantitative PCR in parental D2A1, D2.OR, and D2.1 cells of Esr1. Error bars represent mean±SD. P values were determined by one-way ANOVA with Tukey's post-hoc correction. B. Esr1 and Greb1 gene expression from RNAseq of D2.1 or D2A1 cells. Error bars represent mean±SD. P values between D2.1 and D2A1 were determined by two-tailed ttest. C. Dkk3 expression by Estrogen receptor (ER) positivity in TCGA BC samples. Comparisons were performed by two-tailed t test. D. Dkk3 expression by molecular subtype in TCGA BC samples. Comparisons were performed by one-way ANOVA with Tukey's post-hoc correction.

### DETAILED DESCRIPTION OF THE INVENTION

Methods of inhibiting the growth of dormant cancer cells, methods of inhibiting CD4+T regulatory cells (Tregs), and methods of treating cancer are provided here. These methods are based on the finding described in the Examples that Dickkopf WNT Signaling Pathway Inhibitor 3 (DKK3) is upregulated in dormant cancer cells and that inhibition of DKK3 can alter the T cell microenvironment by altering the number of CD4+Tregs. Some embodiments of the present disclosure provide methods of inhibiting growth of a dormant cancer cell comprising administering an effective amount of a DKK3 inhibitor to a subject in need thereof to inhibit growth of the dormant cancer cell. As used herein, “dormant tumor”, “dormant cells”, “dormant cancer”, “dormant cancer (or tumor) cells”, and grammatical variants thereof may refer to tumors that possess characteristics of delayed relapse. That is dormant tumors may be seemingly successfully treated by first-, second-, third- (etc.) line therapies (that is, successfully treated by an initial therapy) yet some cancer cells persist. In some cases, tumors may go into remission and recur months, years, or decades later. The cancer cells may remain quiescent with little or no detectable replication during the period of dormancy. Dormant tumors may be “hidden” from immune surveillance; that is, the immune system may be unable to detect dormant tumor cells. Dormant tumors induce a CD4/T regulatory cell-shifted T cell profile via secreted factors, primarily the Wnt pathway antagonist, Dickkopf WNT Signaling Pathway Inhibitor 3 (DKK3). Through this mechanism, dormant tumors may survive long-term even in the presence of activated tumor antigen-specific CD8+T cells. Dormant tumor cells may be quiescent or may not be actively replicating. However, as is a hallmark of cancer, dormant cancer cells may have the potential for irregular or uncontrolled growth but are not actively growing irregularly or uncontrollably.

The inventors performed bulk RNAseq to evaluate phenotypic differences and changes from the selective pressure of adaptive immunity to identify a hybrid epithelial/mesenchymal (E/M) and mammary progenitor expression pattern that was maintained long-term in dormant tumors. Pathway analysis also revealed that even later stage dormant tumors displayed a less proliferative phenotype when compared to non-dormant tumors with upregulated Cdkn1b (p27) and reduced expression of genes associated with proliferation. Gene ontology analysis revealed that eight of the top ten upregulated pathways in dormant tumors specifically related to immune activation, and while general markers of T cells (Cd3d, Cd3e, Cd3g) and cytotoxic T cells (Cd8a, Cd8b1) were associated with both dormant and non-dormant tumors, T regulatory cell (Treg) genes (Ctla4, Foxp3) were only associated with dormant tumors. Subsequent analysis indicated increased CD4 T follicular helper cells, CD8 cells, Tregs, and Naïve B cells in dormant tumors compared to non-dormant tumors despite the possibility that these immune cells were less functional. Further analysis suggests that pro-proliferative signaling in tumors is insufficient to counteract strong immune-editing of proliferative cells, and that a period of dormancy followed by growth is potentially a necessary stage for immune escape in these populations. As such, dormant cells may be identified through gene expression data. For example, dormant cells may be identified as cells having an upregulation of genes analogous to Krt8, Krt14, Krt18, Cdh1, Itgb4, Epcam, Ar, Esr1, Erbb2, H2-K1, H2-K2, H2-D1, Itga5, Sparc, Mmp9, Twist2, Snai1, CD44, Notch1, Acta2, Ptn, Cd200, Nrg1, Id4, Cd74, and/or Sox9 and/or a downregulation of genes analogous to Cd274 and/or Myc. Tumors with dormant characteristics may be identified by the genetic markers listed in Table 1 and may be selected for treatment based on these characteristics. Alternatively or in addition, dormant cancers may be similarly identified and/or characterized by measuring gene expression in T cells in the cancer-having subject. For example, a subject with a dormant cancer may possess higher levels of CD4+T reg cells (as compared to a subject without a dormant cancer, for example), higher ratios of CD4+:CD8+T cells, and/or T cells with an increased expression of genes analogous to Ctla4 and/or Foxp3.

The cancer or cancer cells may include but are not limited to a breast cancer, a pancreatic cancer, a prostate cancer, a brain cancer, an ovarian cancer, a liver cancer, and a lung cancer. In some embodiments, the cancer is a breast cancer, and in some embodiments, the breast cancer is a triple-negative breast cancer (TNBC; lacks expression of estrogen receptor (ER), progesterone receptor (PG) and epidermal growth factor receptor (EGFR; HER2), while in other embodiments the breast cancer is estrogen receptor-positive. Among solid tumors, BC has a propensity for delayed relapse with distinct patterns of recurrence based upon subtype. Those with triple negative breast cancer (TNBC), defined by lack of the estrogen receptor (ER), progesterone receptor (PR), and human epidermal growth factor receptor 2 (HER2), are particularly at risk of distant recurrence with a shorter window than other subtypes (33.9% vs 20.4%; 2.6 vs 5 years respectively). In contrast, ER+tumors can recur up to decades after treatment of the primary tumor and have a consistent risk of recurrence over time. The breast cancer may have an increase in Foxp3+T regulatory cells. Foxp3+T regulatory cells may be CD4 T cells. Methods of determining the number of T cells in a sample, which may be referred to as “T cell count”, are well known in the art and may or may not include the methods described herein.

As used herein, “a subject in need thereof” may be understood to mean a subject having a cancer, having had a cancer, or at risk of developing a cancer or cancer recurrence; and the subject may or may not be human. In some embodiments, the subject in need thereof previously received a cancer therapy treatment for a cancer which may include, but is not limited to, a first-line treatment for the cancer. First-line treatments may include, but are not limited to, radiation, chemotherapy, surgery, immunotherapy, gene therapy, hormone therapy, anti-angiogenic therapy, and cytokine therapy. Initial or subsequent cancer treatments may be successful in treating cancer, but they may result in tumor dormancy that is largely attributable to residual tumor cells that enter a state of quiescence or minimal proliferation until some other condition for growth is attained. Many cancer treatments are useful in that their mechanisms of action target proliferative (or actively growing) cells. In addition to being mostly non-proliferative, dormant cancer cells actively communicate with the local stroma to alter the microenvironment and support their own survival. The advent of immunotherapy has highlighted the role of immune cells in an evolving tumor, even during dormancy. The inventors demonstrate that dormant tumors are recognizable by T cells but manage to persist in the presence of activated tumor antigen-specific CD8+T cells before, during, and/or after cancer treatment. To survive cancer treatment, dormant cells induce a CD4/Treg shifted T cell profile via secreted factors and the inventors identified DKK3 as a crucial mediator of this effect. It is understood by those in the art that the cancer therapy treatment will vary depending on certain factors including, but not limited to, disease stage, molecular subtype, and age of the subject. In some embodiments, the cancer therapy treatment for the cancer resulted in remission of the cancer.

DKK3 inhibitors used in the methods herein may be an antibody or antibody fragment. Suitable antibodies may bind DKK3 and at least partially inhibit DKK3 function. Suitable antibodies may include but are not limited to those described in the art, including but not limited to those described in U.S. Patent Publication Number 2021/0340232A1 or other commercially available antibodies that inhibit DKK3 function. In some embodiments, the administration of the effective amount of the DKK3 inhibitor increases CD8 proliferation and/or decreases CD4 proliferation, in particular CD4+Treg cells. Methods of measuring T cell proliferation are known in the art and described in the present examples. Other means of inhibiting DKK3 include but are not limited to those described in the present disclosure such as use of an shRNA specific for DKK3 or other RNA-based inhibitory methods. The DKK3 inhibitor can be administered to the subject via any means known to those skilled in the art and the means of administration will likely be determined by the actual DKK3 inhibitor being administered. For example, DKK3 inhibitors such as antibodies may be administered via injection to help maintain remission of the cancer in individuals previously treated for a primary tumor or cancer. DKK3 inhibitors may also be administered using a gene therapy approach such as the administration of a viral vector capable of expressing an RNA or antibody (affinity reagent) capable of inhibiting DKK3 expression as shown using a lentiviral vector expressing an shRNA specific for DKK3 in the examples.

The cancers may include at least one metastasis. As used herein, “metastasis” and grammatical equivalents may refer to the growth of cancer cells in organs distant from the one in which they originated. Expression of Dkk3 was shown to be higher in primary tumors of BC patients that presented with metastasis, even in ER+tumors which have higher Dkk3 at baseline. As such, Dkk3 expression may be a useful marker in predicting metastatic occurrence and patient populations may be selected by a Dkk3 expression criterium. Metastases may be found throughout the body and includes, but is not limited to, bones, liver, lungs, and brain. In some embodiments, the cancer comprises at least one secondary tumor which may be found throughout the body including, but not limited to, bones, liver, lungs, and brain.

The methods provided here may further comprise administering an immunotherapy. The immunotherapy may comprise an immune checkpoint inhibitor. Immune checkpoint inhibitors which may be used according to the invention are any that disrupt the inhibitory interaction of cytotoxic T cells and tumor cells. These include but are not limited to anti-PD-1 antibody, anti-PD-L1 antibody, anti-CTLA4 antibody, anti-LAG-3 antibody, and/or anti-TIM-3 antibody. The inhibitor need not be an antibody but can be a small molecule or other polymer. If the inhibitor is an antibody, it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GAL9, LAG3, VISTA, KIR, 2B4, CD160, CGEN-15049, CHK1, CHK2, A2aR, and the B-7 family of ligands. Combinations of inhibitors for a single target immune checkpoint or different inhibitors for different immune checkpoints may be used. Several checkpoint inhibitors are commercially available and known in the art. For example, tremelimumab, an anti-CTL4 antibody is available from MedImmune (AstraZeneca) and described in U.S. Pat. No. 6,682,736 and EP Patent No. 1141028; atezolizumab is an anti-PD-L1 available from Genentech, Inc. (Roche) and described in U.S. Pat. No. 8,217,149; ipimilumab, an anti-CTLA-4 available from Bristol-Myers Squibb Co, described in U.S. Pat. Nos. 7,605,238, 6,984,720, 5,811,097, and EP Patent No. EP1212422, among others; pembrolizumab, and anti-PD-1 antibody, available from Merck and Co and described in U.S. Pat. Nos. 8,952,136, 83,545,509, 8,900,587 and EP2170959; nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; tislelizumab available from BeiGene and described in U.S. Pat. No. 8,735,553; among others.

Methods provided herein may further include administering at least one additional cancer therapy. The at least one additional cancer therapy may selected from radiation, chemotherapy, surgery, immunotherapy, gene therapy, hormone therapy, anti-angiogenic therapy, and cytokine therapy.

Another embodiment of the present disclosure provides a method of inhibiting CD4+Treg cells in a subject comprising administering an effective amount of a DKK3 inhibitor to a subject in need of inhibition of a CD4+T reg cell response. After administration of the DKK3 inhibitor the subject has a decrease in Foxp3+T regulatory cells and suitably an increase in the cancer cell specific CD8+T cells. The DKK3 inhibitor may be an antibody or antibody fragment, or an RNA-based DKK3 inhibitor and may be administered directly or via a gene therapy vectored approach. The method may result in increases CD8+T cell proliferation or activation and/or decreases CD4+T cell proliferation following administration of the effective amount of the DKK3 inhibitor.

Yet another embodiment of the present disclosure provides a method of inhibiting cancer recurrence in a subject previously treated for a cancer. The method includes administering an effective amount of a DKK3 inhibitor to the subject following the completion of a cancer therapy in the subject. Previous treatment for the cancer may or may not be complete, and the cancer may or may not be in remission. Treatments for cancer may include, but are not limited to, radiation, chemotherapy, surgery, immunotherapy, gene therapy, hormone therapy, anti-angiogenic therapy, and cytokine therapy. Initial or subsequent cancer treatments may be successful in treating cancer, but they may result in tumor dormancy that is largely attributable to residual tumor cells that enter a state of quiescence or minimal proliferation until some other condition for growth is attained. Many cancer treatments are useful in that their mechanisms of action target proliferative (or actively growing) cells. In addition to being mostly non-proliferative, dormant cancer cells actively communicate with the local stroma to alter the microenvironment and support their own survival. The advent of immunotherapy has highlighted the role of immune cells in an evolving tumor, even during dormancy. The inventors demonstrate that dormant tumors are recognizable by T cells but manage to persist in the presence of activated tumor antigen-specific CD8+T cells before, during, and/or after cancer treatment. To survive cancer treatment, dormant cells induce a CD4/Treg shifted T cell profile via secreted factors and the inventors identified DKK3 as a crucial mediator of this effect. It is understood by those in the art that the cancer therapy treatment will vary depending on certain factors including, but not limited to, disease stage, molecular subtype, and age of the subject. In some embodiments, the cancer therapy treatment for the cancer resulted in remission of the cancer .. The treatment may continue while the cancer is considered to be in remission and delays recurrence of the cancer as compared to a similar subject not administered the DKK3 inhibitor.

The present disclosure also provides methods of treating a subject with cancer by providing a treatment regimen. During the diagnosis or early evaluation of a subject diagnosed with or being diagnosed for cancer a sample of cancer cells from the subject may be obtained and the level of expression of DKK3 in the cancer cells may be determined. A sample from the subject may include any sample containing cancer cells or being evaluated for the presence of cancer cells. Suitable samples include, but are not limited to biopsy tissue or cells, tissue or cells obtained during a surgical procedure whether as a means to remove the tumor or simply evaluate the tumor or cancer, e.g., a tumor resection or needle biopsy. Means of obtaining samples including cancer cells are well known in the art. Means of determining the level of expression of DKK3 or other genes and/or proteins include any methods of measuring protein or gene expression known to those of skill in the art. These methods include but not limited to quantitative rt-PCR, northern blotting, Western blotting, ELISA, and FACs analysis. If present, the cancer may be treated using a standard of care cancer therapy in the subject or the cancer may be surgically removed. For subjects with cancer and wherein the cancer cells are determined to express DKK3, a DKK3 inhibitor may be administered to the subject. In those subjects showing increased expression levels of DKK3 in the cancer cells in the sample and after completion of treatment a DKK3 inhibitor can be administered as described above. Administration of the DKK3 inhibitor to those subjects showing increased expression in the cancer cells may delay recurrence of the cancer or lower the risk or aggressiveness of the recurrence of the cancer.

The term “antibody” refers to a full-length antibody, derivatives or fragments of full length antibodies that comprise less than the full length sequence of the antibody but retain at least the binding specificity of the full length antibody (e.g., variable portions of the light chain and heavy chain), chimeric antibodies, humanized antibodies, synthetic antibodies, recombinantly produced antibodies, as known to those skilled in the art, and produced using methods known in the art. Examples of antibody fragments include, but are not limited to, diabodies, monobodies, Fab, Fab′, F(ab′)₂, scFv, Fv, dimeric scFv, Fd, and Fd. Fragments may be synthesized or generated by enzymatic cleavage using methods known in the art. Antibodies can also be produced in either prokaryotic or eukaryotic in vitro translation systems using methods known in the art. Antibodies may also be referred to herein by their complementarity-determining regions (CDRs), part of the variable chains in antibodies that bind to their specific antigen. Thus, an antibody may be referred to herein by its CDRs of the heavy chain (V_{H} CDR 1, V_{H} CDR 2, and V_{H} CDR 3), and the light chain (V_{L} CDR 1, V_{L} CDR 2, and V_{L} CDR 3) for illustrative purposes. Likewise, an antibody of an IgG class may be referred to by its subclass (e.g., IgG1, IgG2, IgG3, and IgG4). Amino acid sequences are known to those skilled in the art for the Fc portion of antibodies of the respective IgG subclass.

Antibodies herein specifically include “chimeric” antibodies (immunoglobulins), as well as fragments of such antibodies, as long as they exhibit the desired biological activity (U.S. Pat. No. 4,816,567; Morrison et al., Proc. Natl. Acad. Sci. USA 81:6851-6855 (1984); Oi et al., Biotechnologies 4(3):214-221 (1986); and Liu et al., Proc. Natl. Acad. Sci. USA 84:3439-43 (1987)).

“Humanized” or “CDR grafted” forms of non-human (e.g., murine) antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. The term “hypervariable region” when used herein refers to the amino acid residues of an antibody which are associated with its binding to antigen. The hypervariable regions encompass the amino acid residues of the “complementarity determining regions” or “CDRs”. In some instances, framework region (FW) residues of the human immunoglobulin are also replaced by corresponding non-human residues (so called “back mutations”). Furthermore, humanized antibodies may be modified to comprise residues which are not found in the recipient antibody or in the donor antibody, in order to further improve antibody properties, such as affinity. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); and Reichmann et al., Nature 332:323-329 (1988).

“Single-chain Fv” or “sFv” or “scFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et. al., Proc. Natl. Acad. Sci. USA 90:6444-6448 (1993).

The expression “linear antibodies” when used throughout this application refers to the antibodies described in Zapata, et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

Treating cancer will be readily understood by those skilled in the art and includes, but is not limited to, reducing the number of cancer cells or the size of a tumor in the subject, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms or slowing the progression of symptoms, etc. Completion of treatment for cancer may include, but is not limited to, full remission of cancer and/or completion of at least one treatment regimen.

Co-administration, or administration of more than one composition (i.e., an antibody and an immunotherapeutic agent) to a subject, indicates that the compositions may be administered in any order, at the same time or as part of a unitary composition. The two compositions may be administered such that one is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

An effective amount or a therapeutically effective amount as used herein means the amount of a composition that, when administered to a subject for treating a state, disorder or condition is sufficient to affect a treatment (as defined above). The therapeutically effective amount will vary depending on the compound, formulation or composition, the disease and its severity and the age, weight, physical condition and responsiveness of the subject to be treated.

The compositions (i.e., the DKK3 inhibitor and the additional therapeutic agents or checkpoint inhibitory agents) described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, or transmucosal absorption. Thus, the compositions may be formulated as an ingestible, injectable, topical or suppository formulation. The compositions may also be delivered with in a liposomal or time-release vehicle. Administration of the compositions to a subject in accordance with the invention appears to exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological or prophylactic protocol.

The maximal dosage for a subject is the highest dosage that does not cause undesirable or intolerable side effects. The number of variables in regard to an individual prophylactic or treatment regimen is large, and a considerable range of doses is expected. The route of administration will also impact the dosage requirements. It is anticipated that dosages of the compositions will reduce the growth of the cancer at least 10%, 20%, 30%, 40%, 50%, 60%, 70%, 80%, 90%, 100% or more as compared to no treatment or treatment with only the therapeutic agent. It is specifically contemplated that pharmaceutical preparations and compositions may palliate, block further growth or alleviate symptoms associated with the cancer without providing a cure, or, in some embodiments, may be used to cure the cancer and rid the subject of the disease. The effective dosage amounts described herein refer to total amounts administered, that is, if more than one composition is administered, the effective dosage amounts correspond to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

The term “pharmaceutically acceptable carrier” is used herein to mean any compound or composition or carrier medium useful in any one or more of administration, delivery, storage, stability of a composition or combination described herein. These carriers are known in the art to include, but are not limited to, a diluent, water, saline, suitable vehicle (e.g., liposome, microparticle, nanoparticle, emulsion, capsule), buffer, medical parenteral vehicle, excipient, aqueous solution, suspension, solvent, emulsions, detergent, chelating agent, solubilizing agent, salt, colorant, polymer, hydrogel, surfactant, emulsifier, adjuvant, filler, preservative, stabilizer, oil, binder, disintegrant, absorbent, flavor agent, and the like as broadly known in the pharmaceutical art.

It is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Further, as used herein, ranges that are between two particular values should be understood to expressly include those two particular values. For example, “between 0 and 1” means “from 0 to 1” and expressly includes 0 and 1 and anything falling inside these values. Also, as used herein “about” means±20% of the stated value, and includes more specifically values of±10%, ±5%, ±2%, ±1%, and ±0.5% of the stated value.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Dormant Tumors Circumvent Tumor-Specific Adaptive Immunity by Establishing a Treg-Dominated Niche Via DKK3

Approximately 30% of breast cancer survivors deemed ‘free of disease’ will experience locoregional or metastatic recurrence even up to 30 years post initial diagnosis, yet how residual/dormant tumor cells escape immunity elicited by the primary tumor remains unclear. We demonstrate that intrinsically dormant tumor cells are indeed recognized and lysed by antigen-specific T cells in vitro and elicit robust immune responses in vivo. However, despite close proximity to CD8+killer T cells, these examples demonstrate that dormant tumor cells themselves support early accumulation of protective FoxP3+T regulatory cells (Tregs), which can be targeted to reduce tumor burden. These intrinsically dormant tumor cells maintain a hybrid epithelial/mesenchymal state which is associated with immune dysfunction. These Examples demonstrate that the tumor-derived stem/basal gene Dickkopf WNT Signaling Pathway Inhibitor 3 (DKK3) is critical for Treg inhibition of CD8+T cells. We also demonstrate that DKK3 promotes immune-mediated progression of proliferative tumors and is significantly associated with poor survival and immune suppression in human breast cancers. Together, these findings reveal that latent tumors can use fundamental mechanisms of tolerance to alter the T cell microenvironment and subvert immune detection. Thus, targeting these pathways, such as DKK3, may help render dormant tumors susceptible to immunotherapies.

### Introduction

Multiple therapies now exist to treat different molecular subtypes of breast cancer (BC), leading to steady improvement in survival over the past two decades (1). Despite these successes, many survivors (approximately 30%) will eventually experience locoregional or metastatic recurrence, even when there was no clinical evidence of disease after initial therapy (2, 3). Among solid tumors, BC has a propensity for delayed relapse with distinct patterns of recurrence based upon subtype. Those with triple negative breast cancer (TNBC), defined by lack of the estrogen receptor (ER), progesterone receptor (PR), and human epidermal growth factor receptor 2 (HER2), are particularly at risk of distant recurrence with a shorter window than other subtypes (33.9% vs 20.4%; 2.6 vs 5 years respectively) (4). In contrast, ER+tumors can recur up to decades after treatment of the primary tumor and have a consistent risk of recurrence over time (4, 5). Regardless, the time between remission and relapse offers a critical window to eliminate residual tumor cells before developing resistance mechanisms that make recurrent tumors exceedingly challenging to treat.

This phenomenon of delayed relapse, often described generally as tumor dormancy, is largely attributable to residual tumor cells that enter a state of quiescence or minimal proliferation until some other condition for growth is attained (6). To date, multiple mechanisms help explain how these cells enter and exit quiescence. However, relatively little is known regarding their function during the intervening period. Though they are mostly non-proliferative, dormant cancer cells actively communicate with the local stroma to alter the microenvironment and support their own survival (7, 8). Thus, understanding the intrinsic biology of residual, dormant tumor cells is necessary if attempting to eliminate them before recurrence.

The advent of immunotherapy has highlighted the role of immune cells in an evolving tumor, even during dormancy. Organ transplantation provided early evidence that the immune system prevents tumor outgrowth when occult tumors from donated organs began growing in the context of immunosuppressed recipients (9). Further, direct evidence that tumor-specific T cells prevent metastatic outgrowth was also recently described in BC (10, 11), although how residual, dormant tumor cells are not eliminated remains in question. A potential explanation may be that fundamental, developmental mechanisms of tolerance, such as those present at the maternal-fetal interface or during mammary involution, are co-opted to prevent complete tumor elimination (12). While unclear, if immunotherapy is to provide a lasting cure for patients, these overarching processes must be better understood.

Herein, we demonstrate that dormant tumors are recognizable by T cells but manage to persist in the presence of activated tumor antigen-specific CD8+T cells. Our studies reveal that dormancy-competent tumor cells maintain a hybrid epithelial/mesenchymal (E/M) or mammary progenitor-like state in vitro and in vivo. To survive long term, these cells induce a CD4/Treg shifted T cell profile via secreted factors and we identify the Wnt pathway antagonist, Dickkopf WNT Signaling Pathway Inhibitor 3 (DKK3), as a crucial mediator of this effect. Together, the data presented here provide insight into protective barriers that prevent elimination of dormant tumors and lay the foundation for new targets that may be combined with current immunotherapies to provide more durable responses for patients.

### Results

### D2.1 Dormancy is Immune-Independent

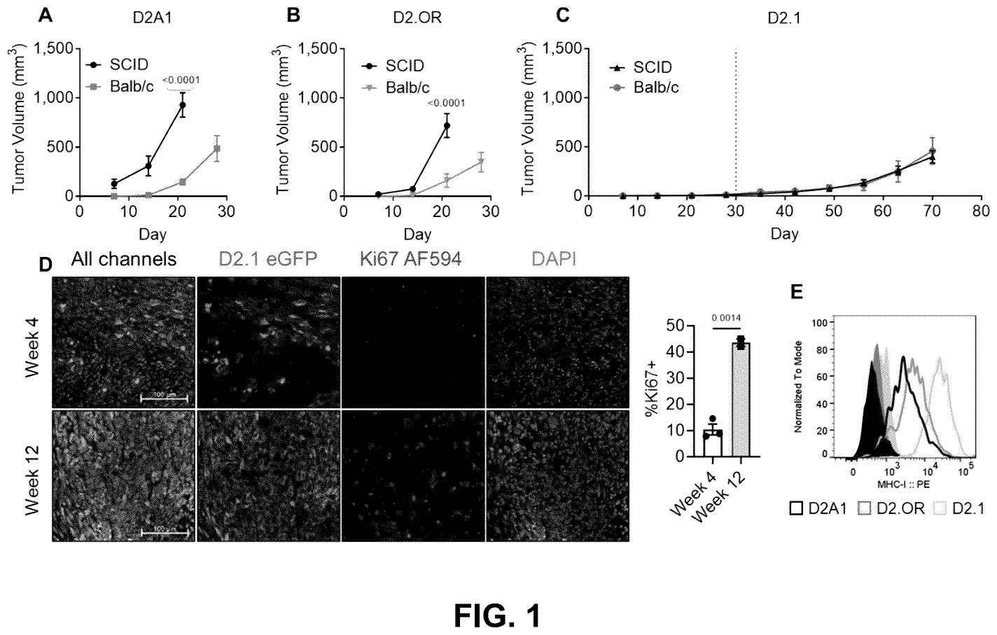

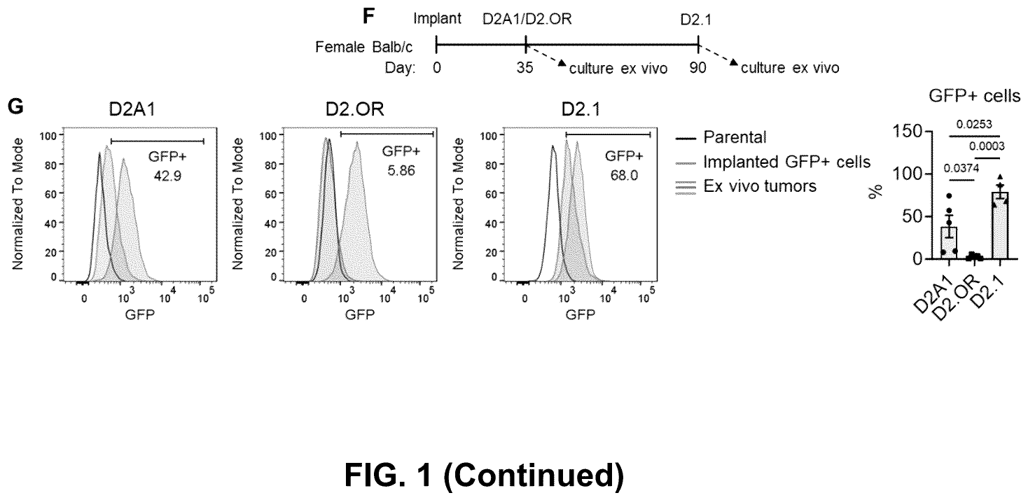

The D2 series of cells, consisting of D2A1, D2.OR, and D2.1 cell lines that arose from D2 hyperplastic alveolar nodules in female BALB/c mice, were used to investigate immunity in tumor dormancy(13, 14). Each cell line is reported to equally extravasate into the lung parenchyma, but only D2A1 rapidly proliferates while D2.OR and D2.1 remain dormant in the metastatic setting (13, 15, 16). Tumor behavior in the presence or absence of adaptive immunity was first determined by implanting these cells in the mammary fat pad (MFP) of BALB/c (immunocompetent) or SCID-beige (deficient in T and B cells and defective NK cells) mice. Both D2A1 and D2.OR tumor growth was significantly delayed in the presence of adaptive immunity (FIG. 1A, B). In contrast, D2.1 tumors were unaffected by T, B, and NK cells (FIG. 1C).

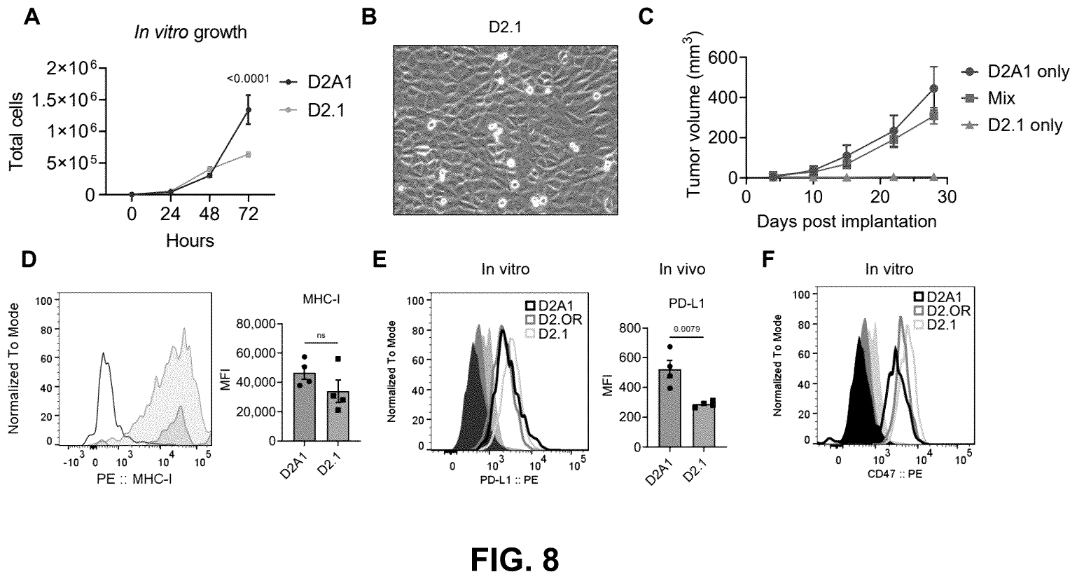

However, when cultured in vitro D2A1 and D2.1 cells proliferate at approximately the same rate until confluence, at which point D2.1 cells become contact inhibited resulting in slowed growth (FIG. 8A, B). To validate the dormant phenotype, D2.1 cells were stably transduced with eGFP by lentiviral vectors and proliferation was assessed in resultant tumors by Ki67 immunofluorescence (IF) at 4 or 12 weeks post MFP implantation in BALB/c animals.

Staining revealed that eGFP+D2.1 tumor cells were minimally positive for Ki67 at 4 weeks, but had significantly elevated Ki67 expression by 12 weeks (FIG. 1D). Co-injection of a 1:1 mix of D2A1 and D2.1 cells also resulted in approximately equivalent tumor growth as D2A1 cells alone, suggesting dormant cells do not induce quiescence in otherwise proliferative cells (FIG. 8C). Collectively, these results demonstrate that D2.1 tumor cells exist as a separate, intrinsically dormant population, unaffected by immune status, that have the eventual capacity to develop into proliferative tumors.

Dormant Tumors are Immunologically Protected from Infiltrating T Cells

Because D2.1 tumor persistence through the dormant phase was unaffected by adaptive immunity, cell surface expression of Major Histocompatibility Complex class I (MHC-I), Programmed death-ligand 1 (PD-L1), and CD47 was investigated as potential mechanisms for dormancy-mediated immune evasion (17, 18). Notably, MHC-I (FIG. 1E; FIG. 8D), PD-L1, and CD47 (FIG. 8E, F) are expressed similarly on D2A1, D2.OR, and D2.1 cells, suggesting that these are unlikely to mediate differences in immune resistance. As all D2 lines express comparable cell surface MHC-I, loss of eGFP expression after in vivo implantation was used as a readout for antigen-specific immune selection (FIG. 1F) (19).

While many D2A1 tumor cells resisted eGFP-specific selection, D2.OR cells displayed the highest sensitivity with almost complete loss of eGFP (FIG. 1G). In contrast, the majority of D2.1 cells maintained eGFP after implantation in immune competent, non-eGFP tolerant BALB/c mice. These results are consistent with the recent finding that GFP-specific T cells (Jedi) are unable to eliminate quiescent sub-populations of mammary tumors despite MHC-I expression (20), and suggest that dormant tumors can be effectively shielded from antigen-specific immunity and selection by other means.

D2.1 Dormant Tumor Cells Have a Hybrid E/M Phenotype and are Enriched in Mammary Progenitor Genes Associated with Late Recurrence in Humans

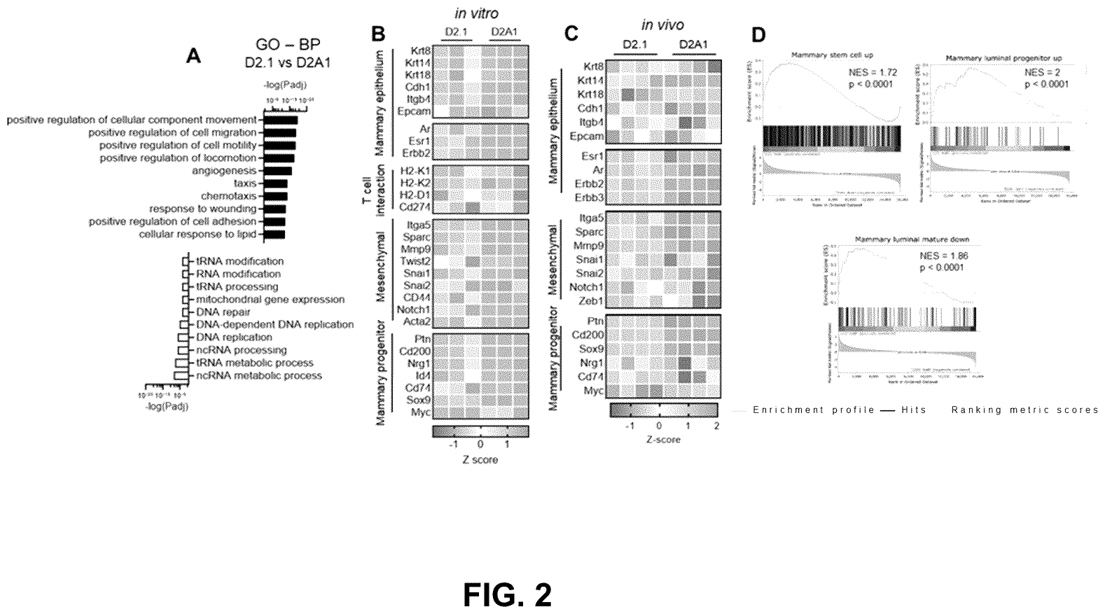

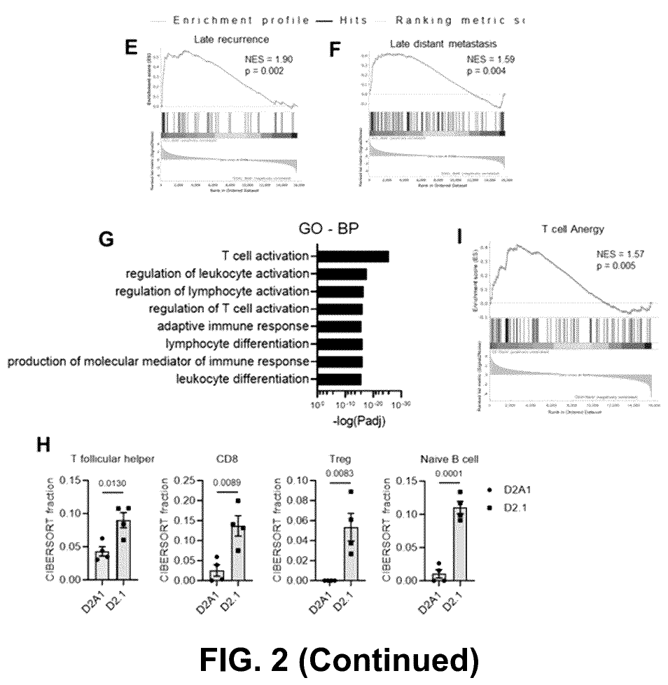

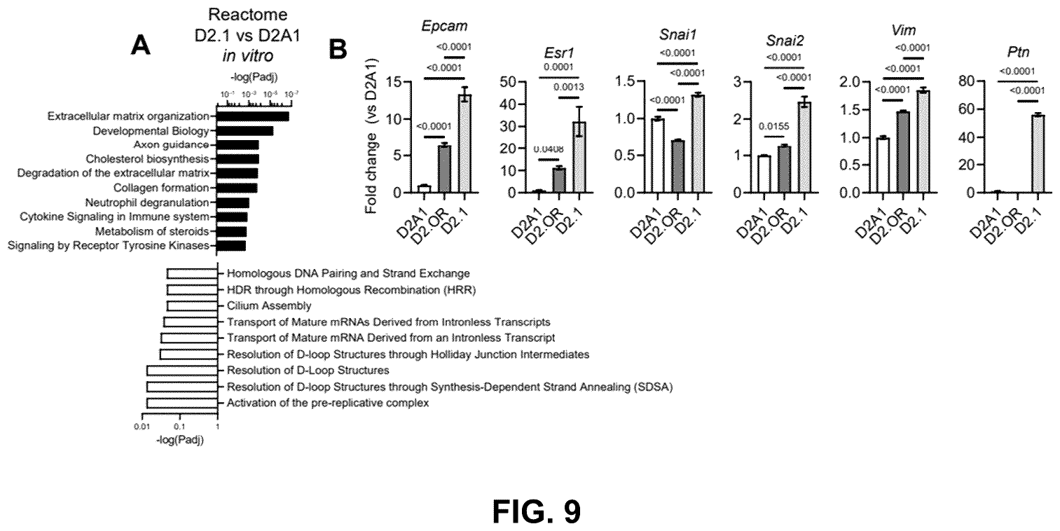

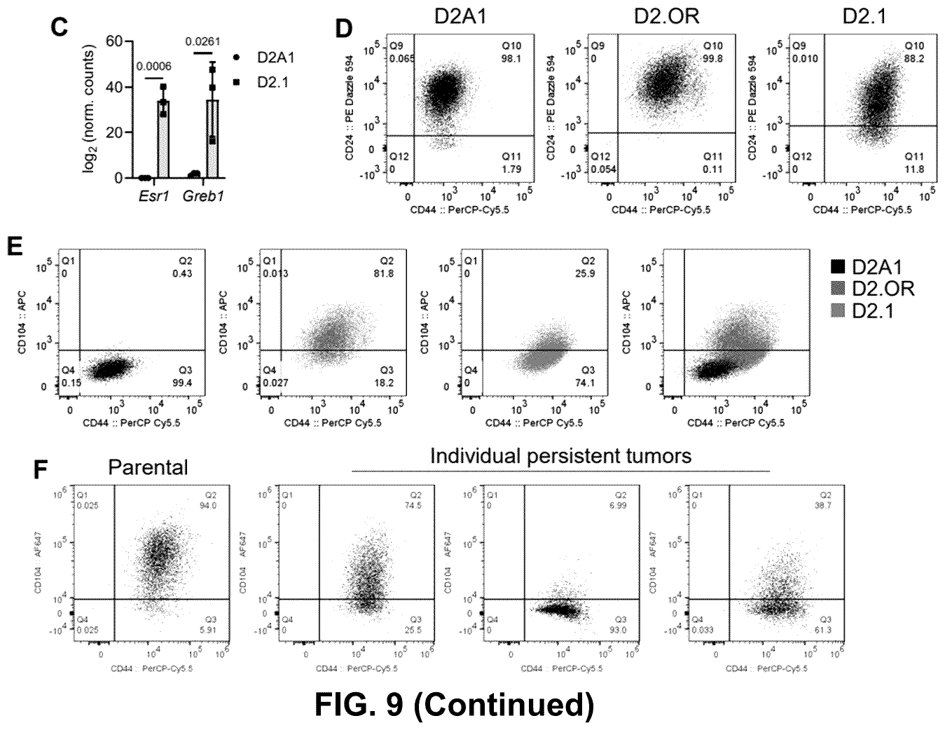

Due to the striking phenotypic differences in vivo between dormancy-competent D2.1 cells and proliferative counterparts we initially performed RNAseq on D2A1 and D2.1 cell lines. The top significantly enriched pathways in D2.1 cells were largely associated with cellular movement and extracellular matrix (ECM) organization with a significant downregulation of metabolic pathways involved in DNA and RNA processing (FIG. 2A; FIG. 9A).

Notably, D2.1 cells expressed higher transcript levels of multiple mammary epithelial makers genes (e.g. Krt8, Krt14, Krt18, Itgb4, Epcam, Cdh1) and genes formerly associated with mammary progenitor cells (e.g. Sox9, CD74, Id4, Nrg1, Ptn, Cd200) (FIG. 2B). Genes that regulate epithelial to mesenchymal transition (EMT) and metastasis were also upregulated, including genes associated with ECM interaction (Itga5, Sparc, Mmp9, Cd44, and Fn1) as well as many transcriptional regulators of EMT (Twist2, Snai1, Snai2, Notch1) (FIG. 2B). Subsequent quantitative PCR confirmed the hybrid E/M phenotype of D2.1 cells which expressed high levels of epithelial and mesenchymal genes compared to both D2A1 and D2.OR cells (FIG. 9B). The ERα gene Esr1 and the ERα target gene Greb1 were also expressed in D2.1 cells (FIG. 9C)—an interesting corollary as human ER+tumors generally display the highest degree of dormancy(5, 21).

In accordance with an overall mammary progenitor-like expression profile, D2.1 cells were found to be CD44hiCD24low/neg compared to D2.OR and D2A1 cells by flow cytometry, with D2.OR cells having the most CD24 expression (FIG. 9D) (22, 23). The presence of increased epithelial and mesenchymal genes could also reflect the described “hybrid Epithelial/Mesenchymal (E/M)” state associated with surface CD44 and CD104 expression (24, 25). Interestingly, surface expression of CD44 and CD104 revealed largely non-overlapping populations between D2A1, D2.OR, and D2.1 cells (FIG. 9E). Because D2.OR tumors are immune-sensitive (FIG. 1G), overt tumors were often rejected in the MFP of BALB/c animals. However, small, residual nodules that re-emerged after approximately 100 days phenotypically resembled D2.1 cells based upon CD44/CD104 expression (FIG. 9F). Altogether, these data indicate that D2.1 cells have hybrid E/M phenotype at baseline which has been associated with metastasis, dormancy, and immunosuppression (24-27), and may represent a particular sub-population that can re-emerge after immune rejection of the tumor at large (as from D2.OR tumors).

### D2.1 Tumor Phenotype is Maintained Throughout Progression

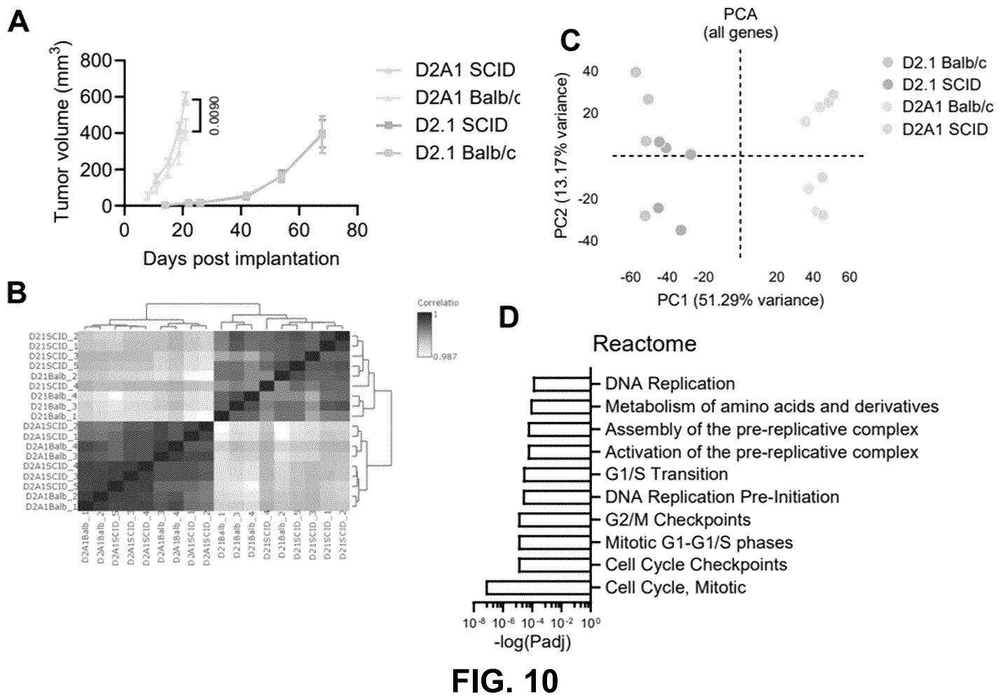

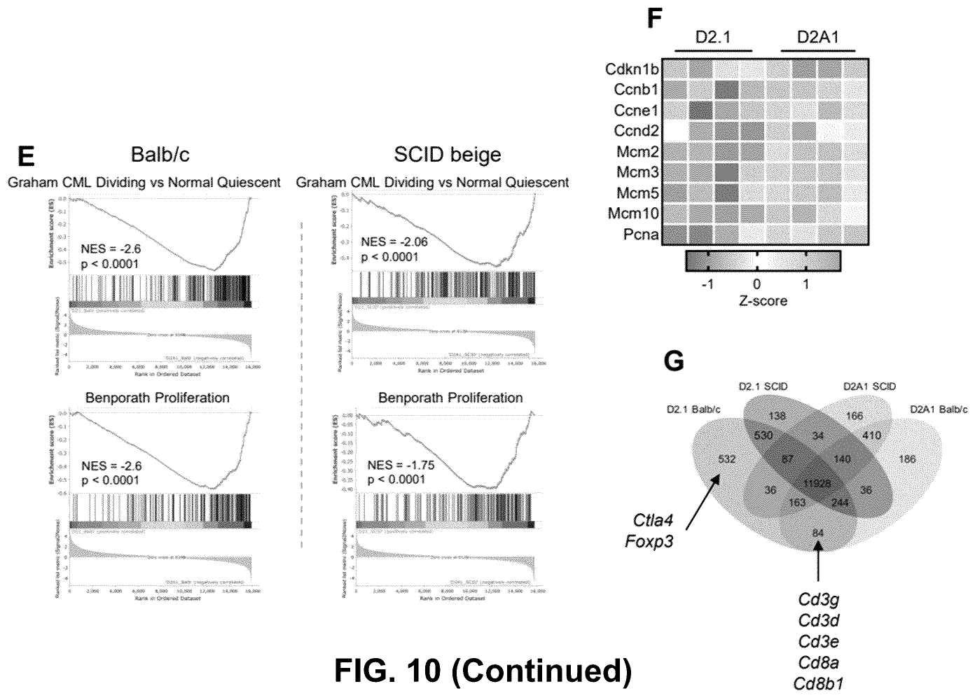

Bulk RNAseq was also performed on MFP D2A1 and D2.1 tumors from both BALB/c and SCID beige mice to evaluate phenotypic differences and changes from the selective pressure of adaptive immunity (FIG. 10A). Overall, D2A1 or D2.1 tumors were largely similar whether in BALB/c or SCID beige mice (FIG. 10B, C). The hybrid E/M and mammary progenitor expression pattern was maintained long-term in D2.1 tumors (FIG. 2C, D) (28). Furthermore, D2.1 tumors were enriched for a genetic profile of late recurrence we previously developed from human BC samples (29), as well as an independent profile of late distant metastasis also generated from human data (30)(FIG. 2E, F; Table 1).

TABLE 1
Gene lists used for GSEA analysis in reference to FIG. 2

| Cheng_Late recurrence | | Mittempergher_Late distant metastasis | | | | | |
|---|---|---|---|---|---|---|---|
| SKAP2 | FAM35A | CXCL13 | IBSP | DPT | CCR6 | IGHA1 | ZNF224 |
| LIMS1 | COL6A3 | CREG2 | TFF1 | ESR1 | CD36 | BACH1 | LOC51152 |
| PLXDC1 | THY1 | LOC348174 | STAC | CDC14A | IGHA1 | IGHV1-69 | NRG1 |
| MAFB | PKD2 | IRF4 | LOC155006 | SPINK4 | GRAP2 | TUB | CEACAM1 |
| SHOX2 | NID2 | SYT13 | WFDC2 | BRUNOL6 | ARMC4 | COLEC11 | HIST1H1D |
| LOH3CR2A | VCAN | IGJ | ADD3 | BAMBI | LOC131873 | CDC42EP3 | GNG2 |
| NNMT | TGFB1I1 | IGHM | PKIA | WBSCR28 | IGHA1 | AIM2 | IGLV6-57 |
| TNC | CALM1 | IGF1 | LOC652791 | RFPL1S | FL139650 | CEACAM5 | TMED6 |
| MRC2 | CXCR7 | CBLN2 | WISP2 | LPHN3 | MAP9 | ADAMTS19 | CCDC65 |
| LAMA4 | IL13RA1 | LMO4 | SLC39A6 | TFAP2B | HS6ST2 | CCL19 | ITM2A |
| DYRK2 | COL1A2 | DIO3 | KLRC1 | IL7 | C7 | CTNND2 | |
| SEMA5A | THY1 | SLC16A7 | SFTPA1 | LOC439949 | FREM1 | CFHR1 | |
| SHOX2 | PDE8A | CH25H | OMD | KLF5 | SLC40A1 | CXCL9 | |
| SNAI2 | ATP2B1 | SELE | OGN | PTGER4 | GJB6 | KIAA1822L | |
| SEC23A | ITGA5 | PITX2 | LOC644488 | CPB1 | XCL1 | LOC652159 | |
| GNS | RABGGTA | BAAT | TFF1 | C8ORFK32 | WNT10A | TMEM119 | |
| HTRA1 | LRRC32 | TNFSF15 | C21orf34 | KLRB1 | PPP2R2C | VGLL3 | |
| DOEF1IT1 | CDKN1A | BHLH65 | NOPE | MGAT56 | DSCR6 | POU2AF1 | |
| LAMB1 | POSTN | FABP4 | MYCBPAP | CA14 | FLJ39370 | LAX1 | |
| COL5A1 | TWIST1 | LUZP2 | CFH | ZBTB16 | C10orf30 | SPFH2 | |
| JAG1 | ATP2B1 | KLK12 | CACNA2D2 | CD96 | CDO1 | CSMD2 | |
| COL1A1 | MSPRBP1 | IGHA1 | TNN | HP | LOC645733 | ASPN | |
| EMLIN1 | ADAM9 | TNFRSF17 | ADD3 | SLC23A1 | IGHA1 | CNTN4 | |
| PHTF2 | DYRK2 | CTNND2 | LOC440361 | MFAP4 | XG | ZBTB12 | |
| COL5A1 | HSPG2 | SLC39A6 | CA3 | LOC390712 | ABCA6 | IGF1 | |
| CLEC11A | TPP1 | WFDC2 | HIST1H2AL | GREM2 | TSPAN13 | WASF3 | |
| JAG1 | AEBP1 | GRIA2 | NRG2 | NBPF4 | HAVCR1 | SH2D1A | |
| ANGPTL2 | MRPL52 | MAP9 | MMP3 | ANK1 | NELL2 | KCNB1 | |
| COL3A1 | | GALNT3 | RASSF8 | LOC644846 | IGKV1-5 | PVRL3 | |

Pathway analysis also revealed that even later stage D2.1 tumors displayed a less proliferative phenotype when compared to D2A1 tumors in both Balb/c and SCID beige animals (FIG. 10D, E) with upregulated Cdknlb (p27; a common indicator of tumor dormancy(20,31)) and reduced expression of genes associated with proliferation (FIG. 10F). Somewhat surprisingly, GO analysis revealed that eight of the top ten upregulated pathways in D2.1 tumors specifically related to immune activation (FIG. 2G), and while general markers of T cells (Cd3d, Cd3e, Cd3g) and cytotoxic T cells (Cd8a, Cd8b1) were associated with tumors from all BALB/c animals, T regulatory cell (Treg) genes (Ctla4, Foxp3) were only associated with D2.1 tumors in immunocompetent animals (FIG. 10G). Subsequent CIBERSORT analysis indicated increased CD4 T follicular helper cells, CD8 cells, Tregs, and Naïve B cells in D2.1 compared to D2A1 tumors (FIG. 2H) despite the possibility that these immune cells were less functional (FIG. 21).

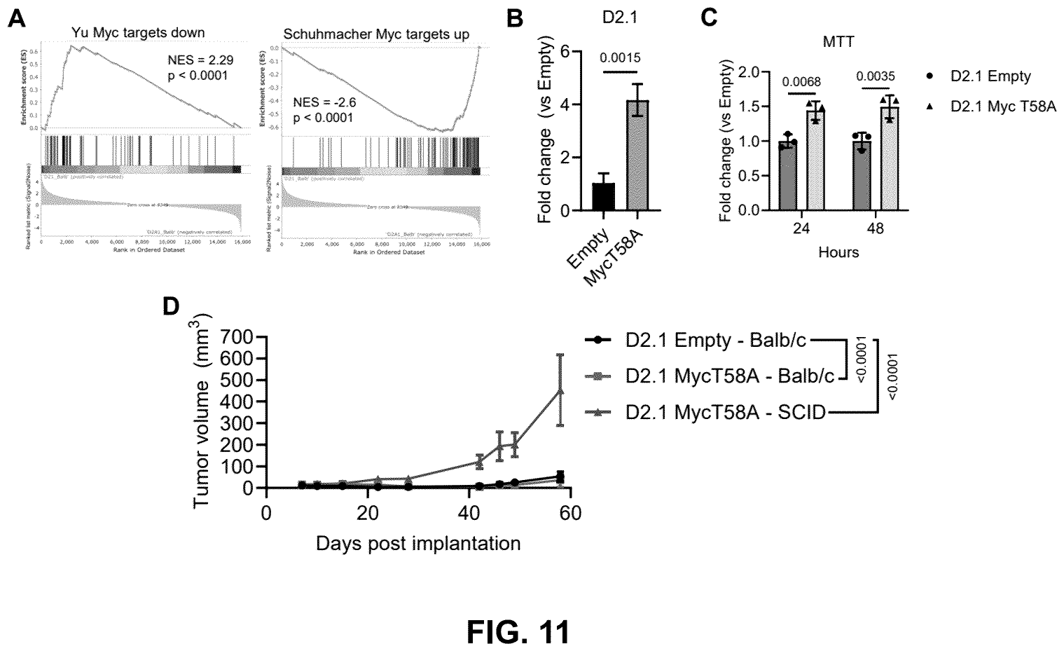

As the Myc oncogene was significantly upregulated in D2A1 tumors (FIG. 2B,C; FIG. 11A), we speculated that the dormant phenotype of D2.1 cells could be reversed by Myc stimulated proliferation. To test this, we expressed a stabilized form of Myc (T58A) (32) in D2.1 cells (FIG. 11B). While Myc-T58A expression enhanced proliferation in vitro and in SCID beige mice it did not translate to enhanced tumor burden in immune competent BALB/c mice (FIG. 11C,D). This suggests pro-proliferative signaling in tumors is insufficient to counteract strong immune-editing of proliferative cells, and that a period of dormancy followed by growth is potentially a necessary stage for immune escape in these populations.

### Dormant Mammary Tumors Have High Levels of Infiltrating Foxp3+T Regulatory Cells

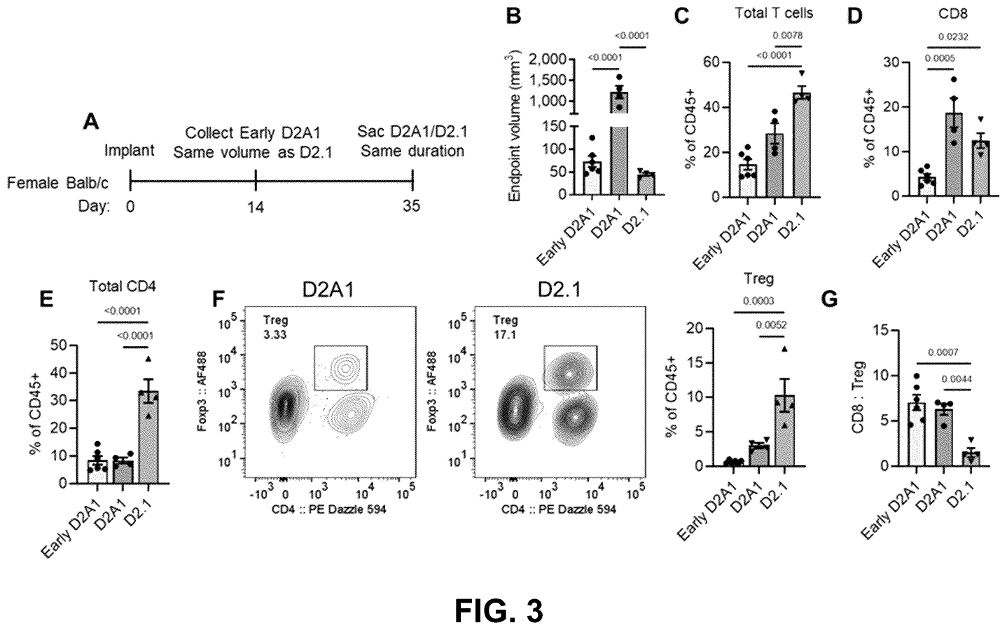

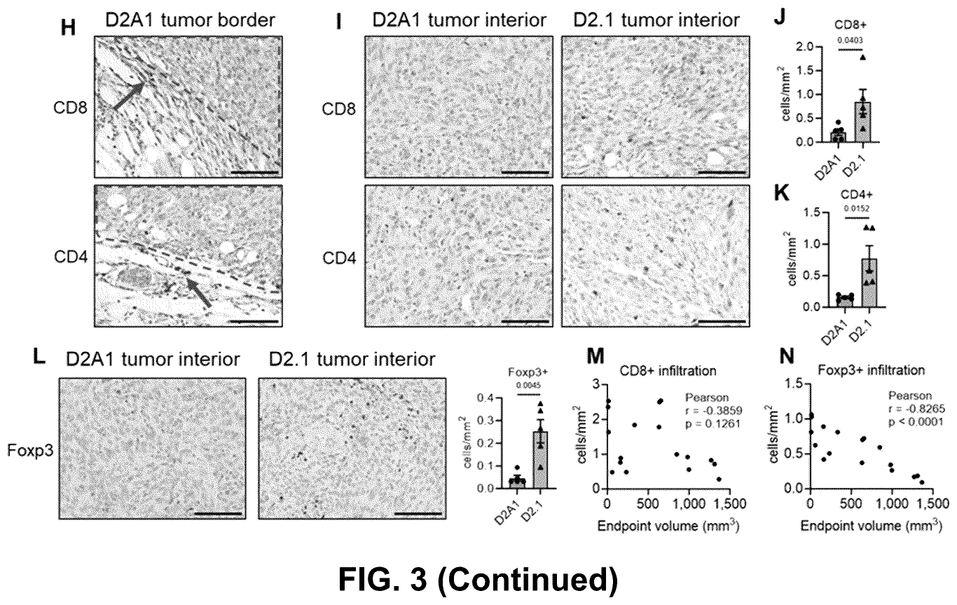

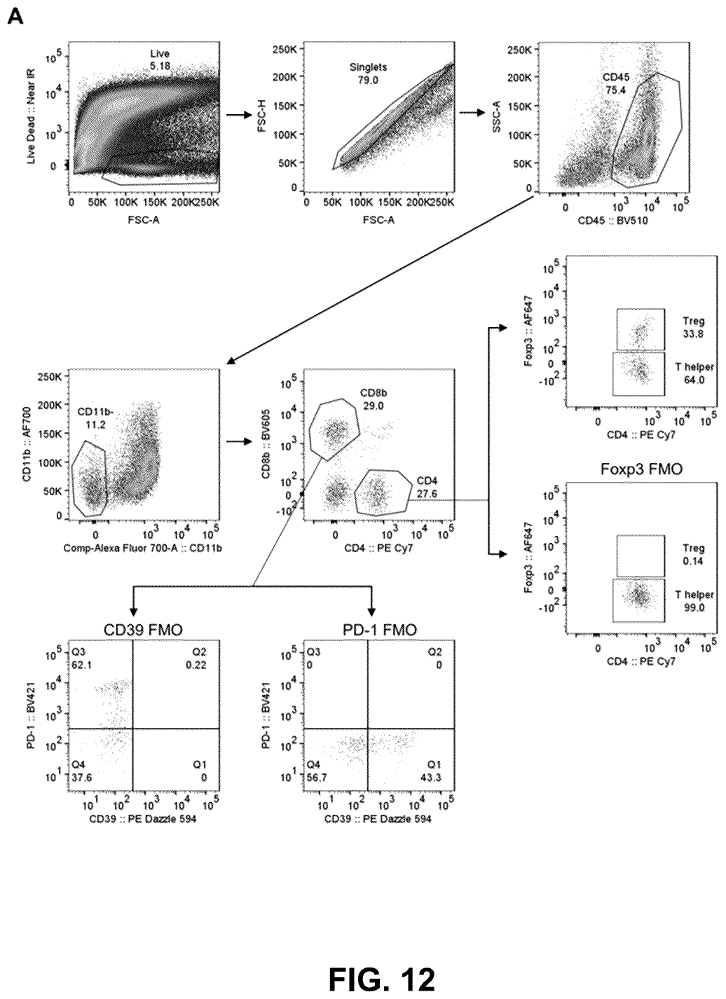

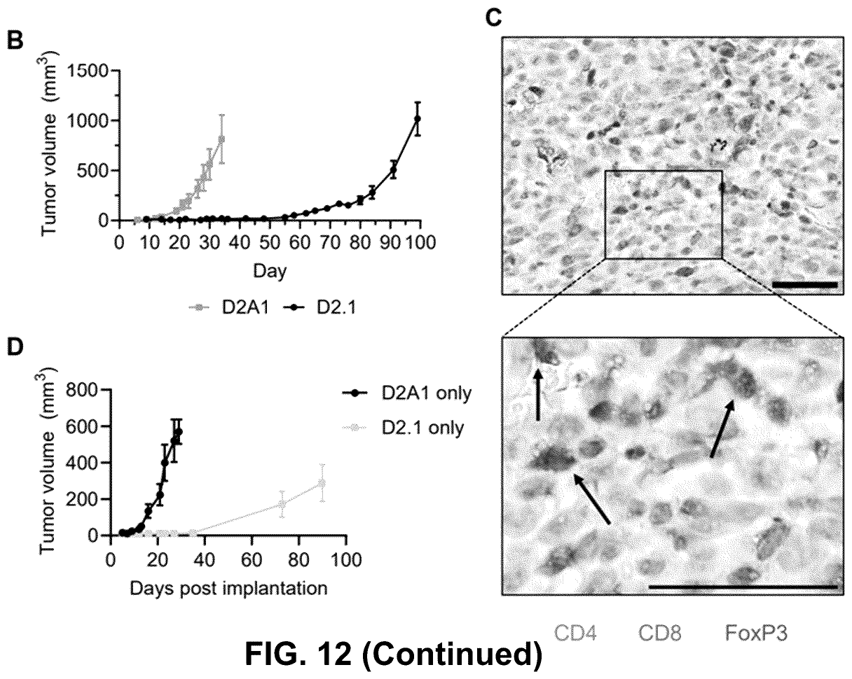

In light of the immune-independent nature of D2.1 latency the presence of immune-related genes in D2.1 tumors was striking. Therefore, D2.1 cells were orthotopically implanted and collected after 35 days for immune analysis, and D2A1 tumors were either collected early at the same final size as D2.1 tumors (14 days post-implantation) or after the same total duration (35 days post-implantation) to ensure that potential differences were not merely due to tumor burden (FIG. 3A, B). Interestingly, flow cytometry revealed that D2.1 tumors indeed contained significantly more total T cells than D2A1 tumors of the same volume or time in vivo (FIG. 3C). While large D2A1 tumors had more infiltrating CD8+T cells in comparison to both small D2A1 tumors and D2.1 tumors (FIG. 3D), D2.1 tumors harbored more total CD4 cells than D2A1 cells at either stage (FIG. 3E). Critically, the Foxp3+CD4+Treg population was significantly elevated in D2.1 tumors (FIG. 3F), ultimately resulting in a decreased CD8:Treg ratio compared to all D2A1 tumors (FIG. 3G). To ascertain the spatial relationship of T cells with tumor cells and determine if differences in T cell profiles were maintained through tumor progression, D2.1 tumors were also collected at a later stage (100 days post-implantation) (FIG. 12B). Staining for CD4+and CD8+T cells revealed that both collected at the tumor/stromal interface in size-matched D2A1 tumors (FIG. 3H; a feature observed in approximately 26% of TNBCs(33)). However, endpoint D2.1 tumors were highly infiltrated with both CD4+and CD8+T cells (FIG. 3I) resulting in a significantly increased interior T cell density in comparison to similarly sized D2A1 tumors (FIG. 3J, K). Furthermore, D2.1 tumors harbored a significant population of infiltrating FoxP3+cells, which likely represents the same CD4+FoxP3+Treg population observed via flow cytometry based upon multicolor IHC (FIG. 3L; FIG. 12C). No correlation in CD8+cell density was observed collectively across D2.1 tumors of multiple timepoints/sizes (FIG. 3M), however Foxp3+cell infiltration was significantly correlated with early, smaller tumors (FIG. 3N). Thus, dormant tumors do not necessarily restrict T cell infiltration even after beginning to proliferate, indicating that changes in T cell function may be more relevant than proximity. Additionally, these data suggest that Treg induction is an early and necessary event to establish the dormant tumor niche for long-term survival.

### Tumor/Antigen-Specific CD8+Cells Do Not Prevent Dormant Tumor Progression In Vivo

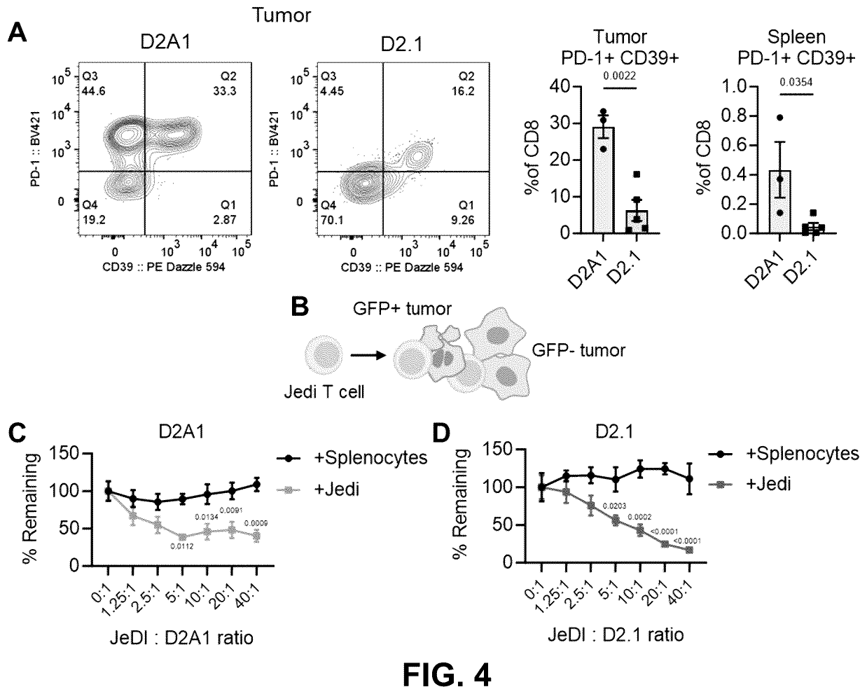

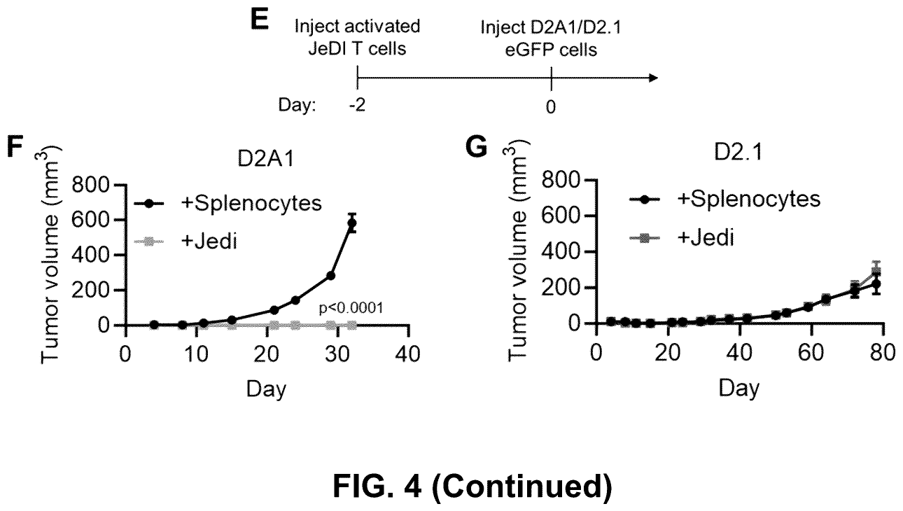

Primary mammary tumors were recently reported to induce CD8+T cells (CD39+PD-1+) that systemically control metastatic dormancy in the lungs in the 4T07 model (10). Contrastingly, analysis of MFP D2A1-eGFP and D2.1-eGFP tumors (FIG. 12E) revealed that D2A1 tumors contained significantly more CD39+PD-1+CD8+T cells than D2.1 tumors, with a similar trend seen systemically in the spleens of tumor-bearing animals (FIG. 4A). Absent a strong, de novo tumor-specific T cell response, we next investigated if D2.1 persistence could be overcome with potent and systemic antigen-specific T cells. To exclude the role of the tumor microenvironment, killing assays were initially performed in vitro to assess the cytotoxic effect of antigen-specific CD8+T cells against D2.1 and D2A1 cells. Just eGFP Death Inducing (Jedi) T cells, which express a H-2Kd restricted T cell receptor (TCR) for GFP₂₀₀₋₂₀₈ (FIG. 4B) (34), were activated/expanded ex vivo and cultured with D2A1 or D2.1 eGFP cells. Both D2A1 and D2.1 cells were equivalently lysed by Jedi cells thereby verifying that both are susceptible to direct killing by activated Jedi CD8+T cells (FIG. 4C, D). Adoptive transfers were then performed to determine if externally activated antigen-specific CD8+T cells could prevent/limit eventual D2.1 tumor outgrowth. Activated Jedi cells were injected intravenously into naïve C57B1/6×BALB/c F1 mice and two days later D2A1 or D2.1 cells expressing eGFP were implanted into the MFP (FIG. 4E). While D2A1 tumor outgrowth was completely prevented by Jedi T cells compared to control T cells (FIG. 4F), D2.1 tumors were unaffected by Jedi T cells in comparison to controls (FIG. 4G). These results indicate that even highly activated antigen-specific CD8+T cells are incapable of preventing establishment and eventual growth of dormant D2.1 tumors in vivo.

### D2.1 Cells Secrete Factors That Induce CD4+and Foxp3+T Regulatory Cells

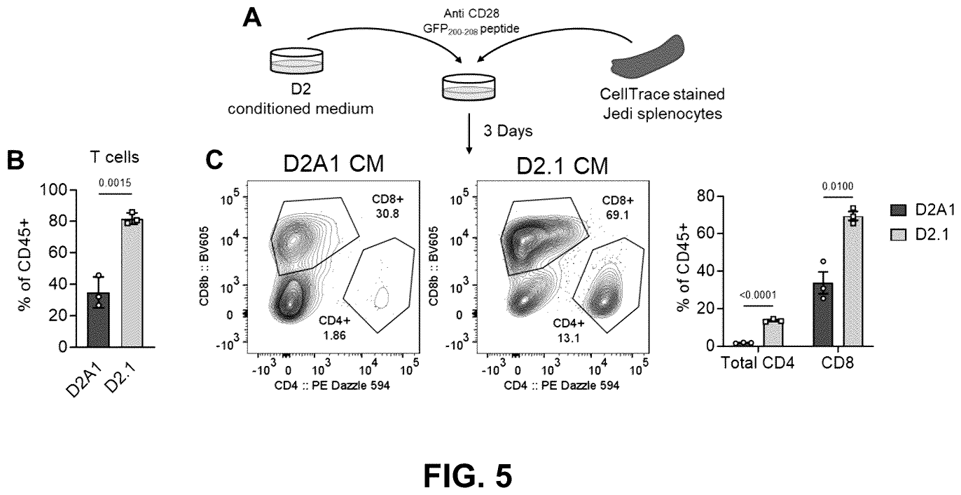

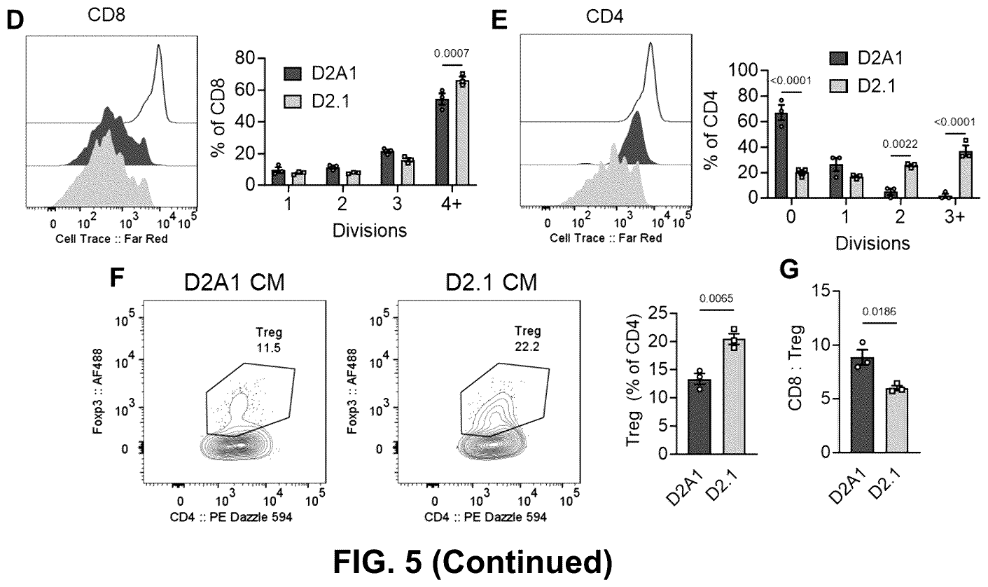

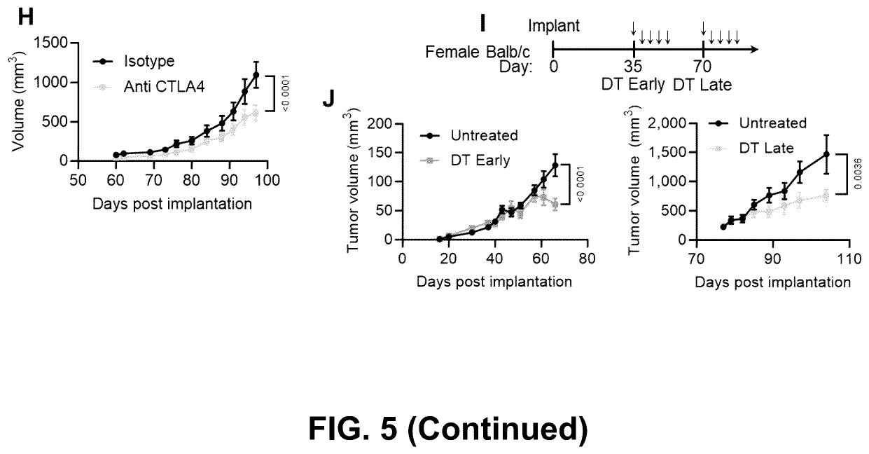

Increasing evidence suggests that epithelial/mesenchymal plasticity, as observed in D2.1 cells, is associated with an overall immunosuppressive microenvironment(27). However, because CD8+T cells were distinctly present in but unable to eliminate latent tumors, we investigated if tumor cells themselves altered T cell function directly. The effect of the tumor secretome on CD8+T cell activation/proliferation was initially to be tested by culturing splenocytes from transgenic Jedi mice with tumor-derived conditioned medium (CM) (35). Whole Jedi splenocytes were stained with Cell Trace dye and stimulated with GFP₂₀₀₋₂₀₈ peptides/anti-CD28 antibodies in the presence of D2A1 or D2.1 CM for 3 days (FIG. 5A). Overall, D2.1 CM resulted in significantly more T cells than D2A1 CM (FIG. 5B), including both CD8+(which was stimulated via peptide) and CD4+cells (FIG. 5C). Cell Trace staining indicated that CD8+T cells indeed underwent more divisions in D2.1 CM compared to D2A1 CM (FIG. 5D), however CD4+T cells displayed more significantly enhanced proliferation in D2.1 CM in comparison to D2A1 CM (FIG. 5E).

Further experiments revealed a significant increase in Tregs within the CD4 population (FIG. 5F), ultimately resulting in a decreased CD8:Treg ratio in D2.1 CM compared to D2A1 (FIG. 5G). Thus, these data suggest that the disparity between D2A1−and D2.1-induced T cell responses in vivo are likely more attributable to dormant tumor cell promotion of CD4+Treg proliferation and/or differentiation via soluble factors.

T regulatory Cells Protect D2.1 Tumors

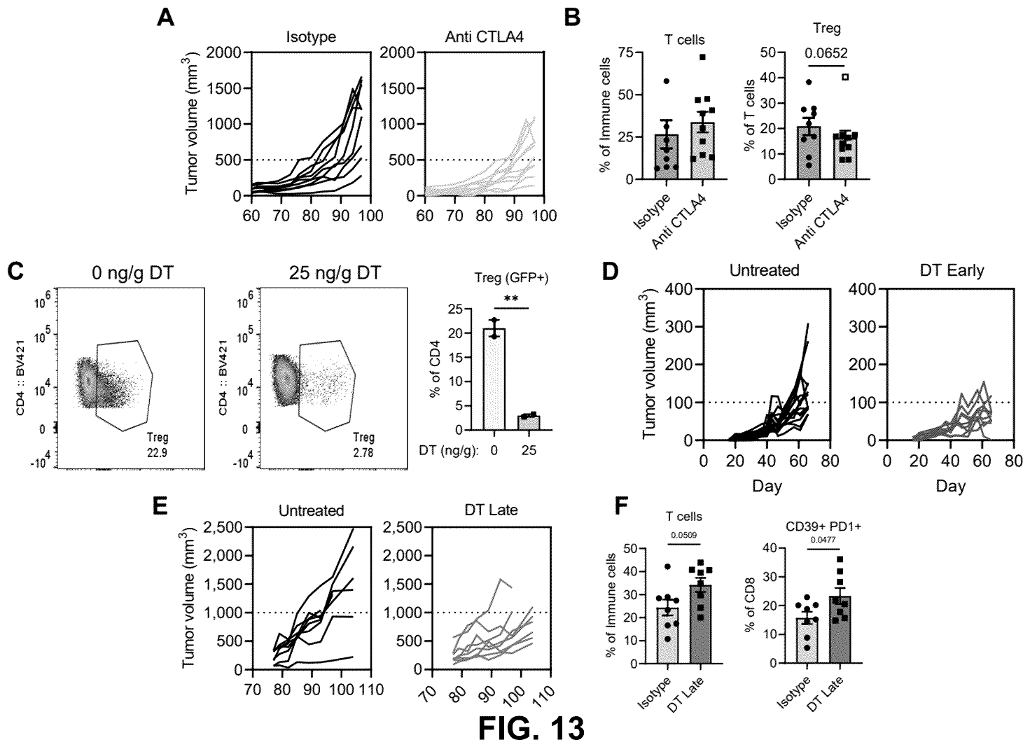

Because CD8+T cell functionality was ostensibly restricted in D2.1 tumors with an associated increase in Tregs, we estimated that Tregs were a more central determinant of immunosuppression in the dormant tumors. Given the ability of anti-CTLA4 IgG2A/B antibodies to deplete intra-tumoral Tregs (36, 37), we first treated mice bearing D2.1 tumors bi-weekly with anti-CTLA4 (clone 9D9, 200 ug/mouse) or isotype controls upon reaching ˜150 mm³. This treatment significantly reduced tumor growth (FIG. 5H; FIG. 13A) with a modest reduction of intra-tumoral Tregs (although total T cell levels were unchanged) (FIG. 13B). While these results suggested the importance of Tregs in dormant tumors, anti-CTLA4 antibodies can alter co-stimulation, thus we wanted to validate these findings in a more specific Treg model. We therefore implanted D2.1 cells into FoxP3-DTR mice, in which Tregs can be ablated through Diptheria Toxin (DT) administration (FIG. 13C).

Tumor bearing animals were treated with DT every four days beginning at an early time point (day 35) or late time point (day 70) with a total of five doses each (FIG. 5I). Notably, both early and late DT treatment resulted in significant reduction of tumor growth (FIG. 5J; FIG. 13D, E). Late DT treated and control tumors were analyzed upon euthanasia which showed an increase in infiltrated T cells (FIG. 13F), including an increase in activated CD39+PD-1+CD8+T cells. Altogether, these data indicate that despite high CD8+T cell infiltration, the concomitant presence of Tregs in D2.1 tumors restricts anti-tumor immunity and enables tumor persistence.

### DKK3 is Crucial for D2.1 Tumor Persistence

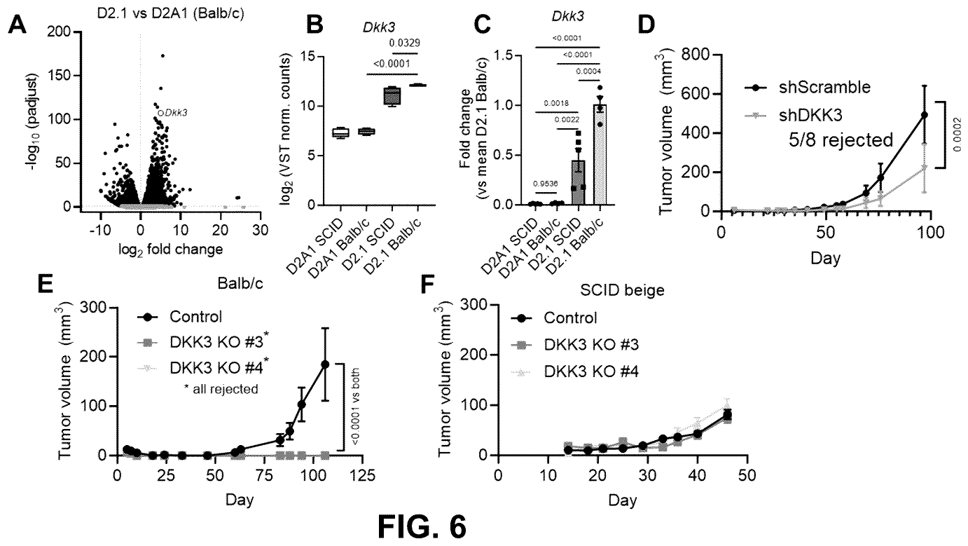

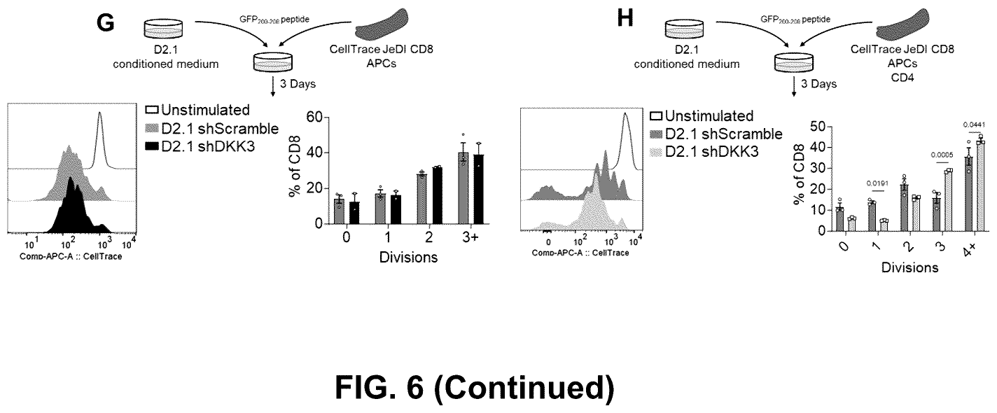

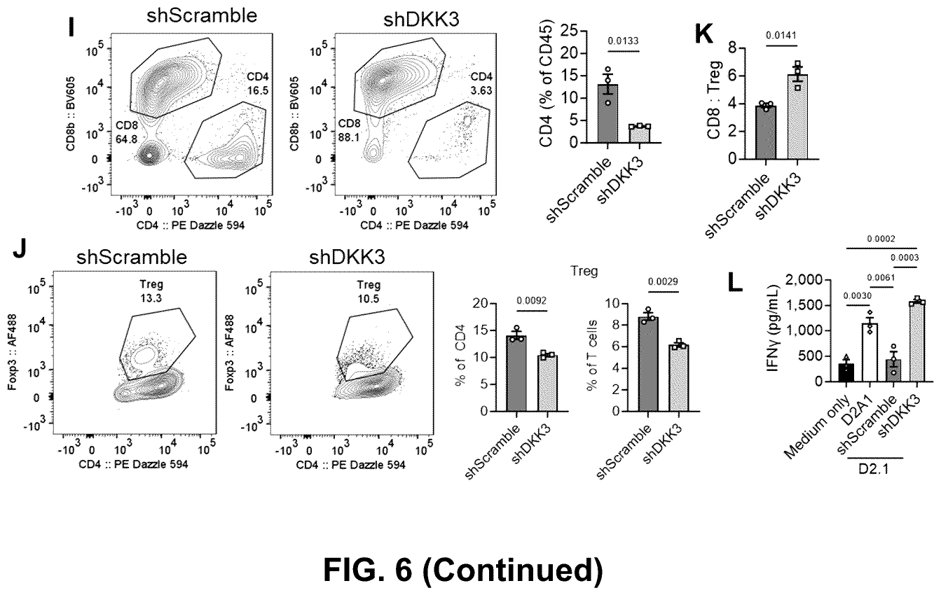

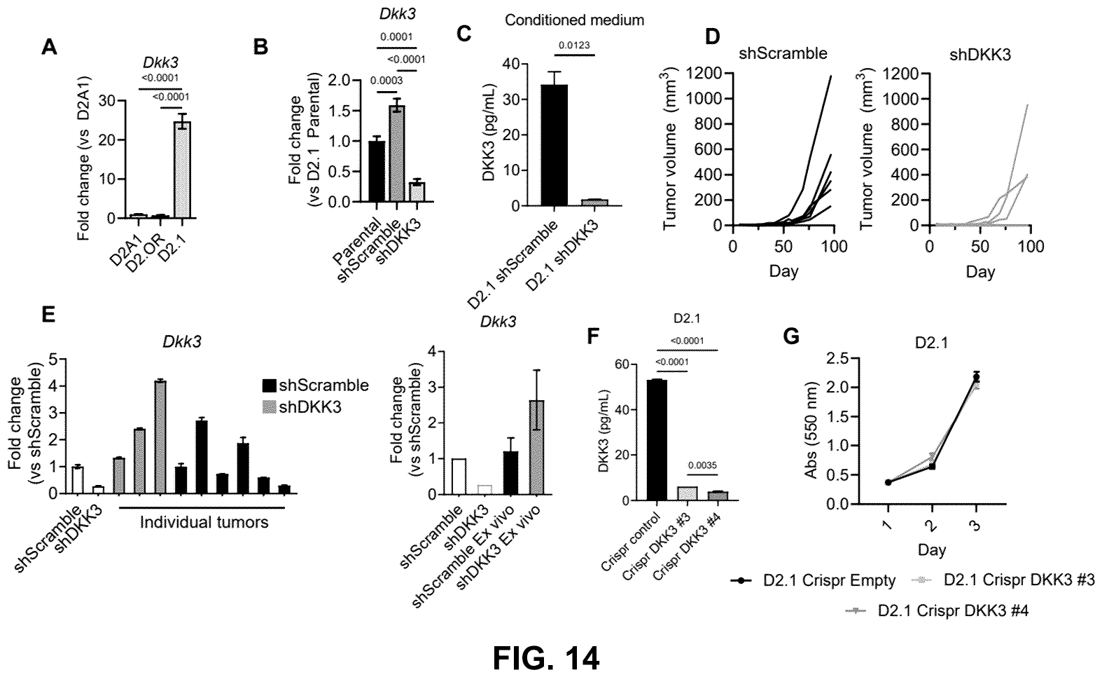

Knowing that tumor-derived secreted factors were sufficient to regulate the T cell landscape we further investigated potential soluble mediators of this effect. The hybrid E/M and mammary progenitor-like signature of D2.1 tumors was typified by high expression of Dickkopf-related protein 3 (DKK3), a Wnt signaling modulator that is preferentially expressed by CD44+CD24−epithelial stem/progenitor cells within the human mammary gland and is generally enriched across stem cell or “immune privileged” niches, making it an attractive candidate (FIG. 6A) (38, 39). Interestingly, DKK3 expression was even greater in D2.1 tumors in immune-competent animals but not D2A1 (FIG. 6B, C). Elevated DKK3 expression in D2.1 cells compared to D2A1 or D2.OR cells was confirmed via quantitative PCR (FIG. 14A) and its function was assessed after stable shRNA knockdown (k/d) in D2.1 cells (FIG. 14B, C).

Upon MFP implantation, shDKK3 D2.1 tumors displayed significantly reduced growth in vivo compared to shScramble controls with the majority being completely rejected (5/8 mice; FIG. 6D; FIG. 14D). Subsequent analysis of tumor cells after expansion ex vivo confirmed that non-rejected shDKK3 tumors regained DKK3 expression—likely explaining persistence of these tumors (FIG. 14E). To validate these results, two independent Crispr DKK3 knockout (KO) D2.1 clones were generated (FIG. 14F, G). When implanted into immunocompetent animals DKK3 KO cells were completely rejected (FIG. 6E) whereas in SCID beige animals DKK3 KO cells yielded tumors equivalent to control cells (FIG. 6F). Thus, DKK3 expression indeed appears to be a necessary component for immune-escape and long-term survival during the dormant phase.

### Tumor-Derived DKK3 Regulates Tregs To Inhibit CD8+T Cell Function

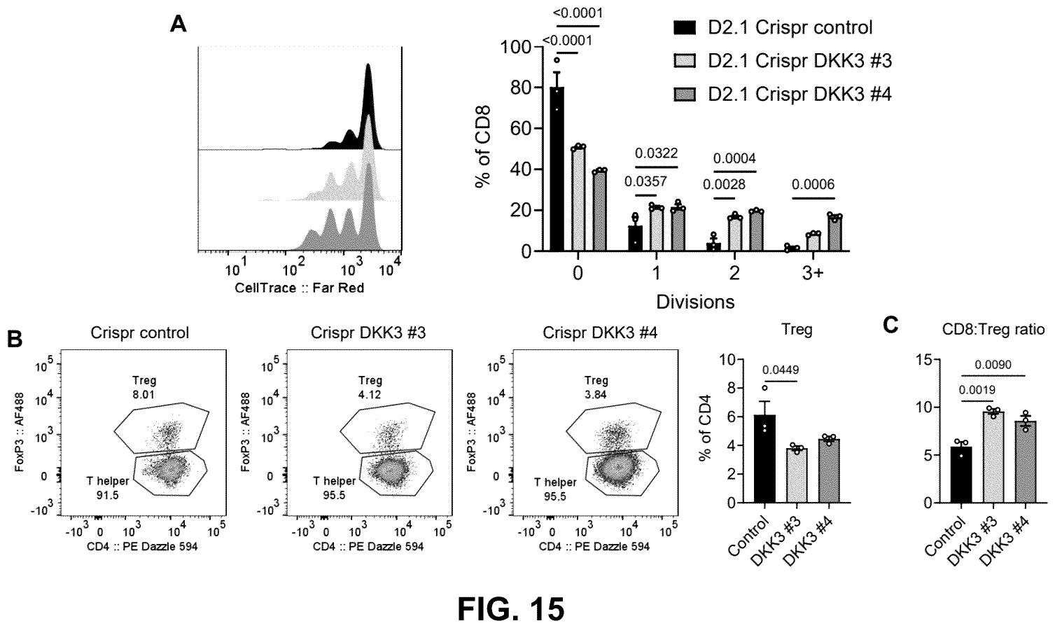

The mechanistic underpinnings of DKK3-mediated immune evasion were further validated ex vivo by stimulating isolated CD8+Jedi T cells mixed with antigen presenting cells (APCs) in D2.1 shScramble or shDKK3 CM. Unexpectedly, no difference in CD8+T cell proliferation was detected between D2.1 shDKK3 CM compared to control (FIG. 6G). However, additional experiments using a mix of CD8+Jedi cells, CD4+cells, and APCs revealed significantly increased total T cells in shDKK3 CM compared to shScramble (FIG. 6H) with increased CD8+T cell proliferation, suggesting an indirect effect of DKK3 on CD8+T cells. Consistent with this observation, the CD4+T cell fraction was significantly reduced in DKK3 silenced D2.1 CM (FIG. 6I) with a decrease of CD4+Foxp3+Tregs (FIG. 6J) yielding an increased CD8:Treg ratio (FIG. 6K). Similar results were also observed when using CM from DKK3 Crispr KO cells (FIG. 15A-C). As a proxy for immune activation, we also assessed the level of IFNγ in media by ELISA and found that DKK3 k/d resulted in increased IFNγ compared to control D2.1 CM and comparable to culture in D2A1 CM (FIG. 6L). Altogether, these experiments support the previous data and implicate CD4+T cells, particularly Tregs, as a target of D2.1 regulation via secretion of DKK3, with secondary effects on CD8+cells.

DKK3 is Associated with Poor Survival and Immune-Evasion in Human BC

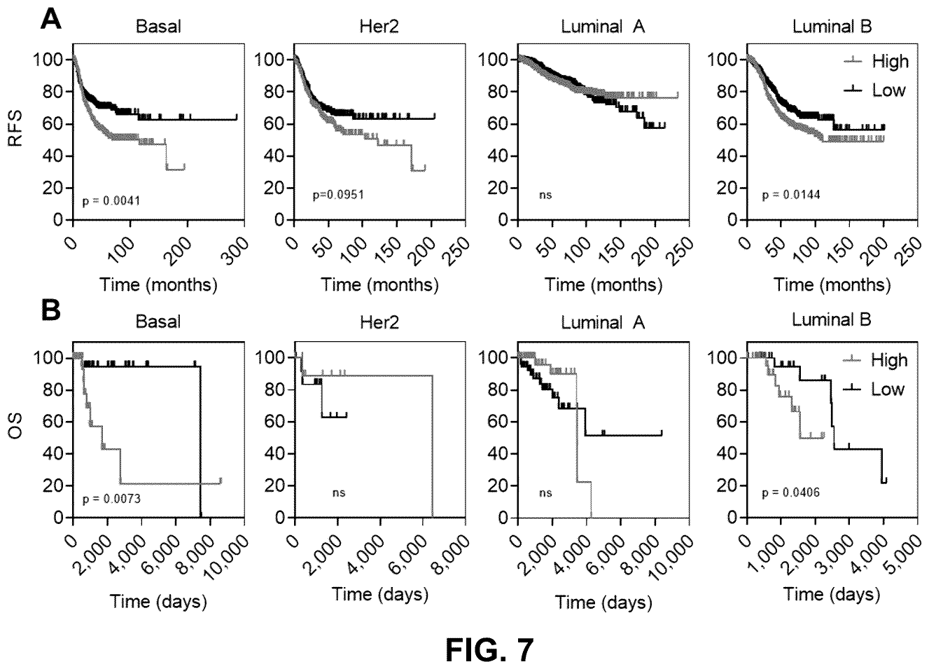

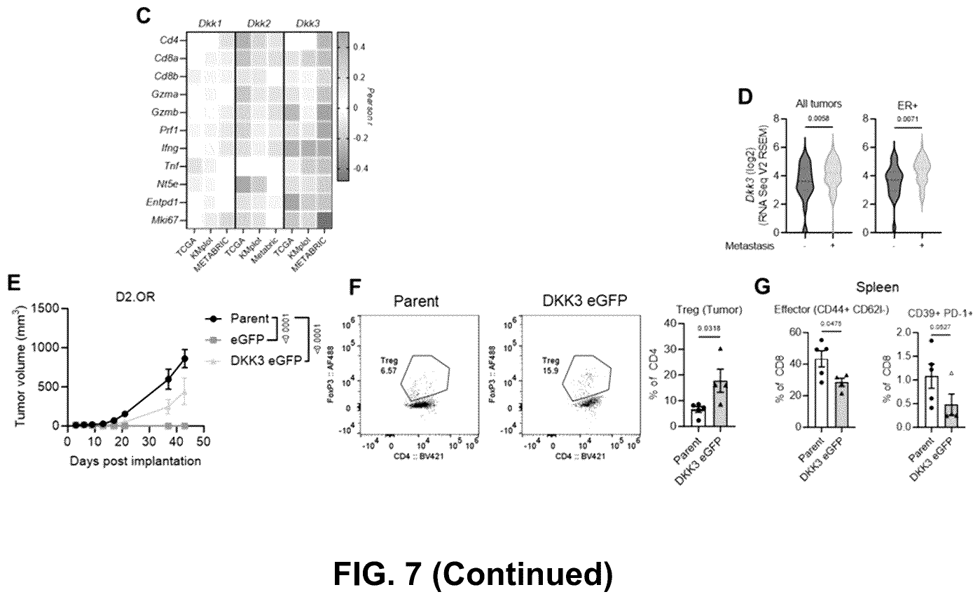

Given the experimental evidence of DKK3-modulated immune function, DKK3 was assessed in BC datasets obtained from clinical samples to determine relevance of this pathway in humans. In BC, we found that high Dkk3 expression was significantly correlated with poor survival in Basal and Luminal B tumors in both the KMPlotter and TCGA datasets (FIG. 7A,B).

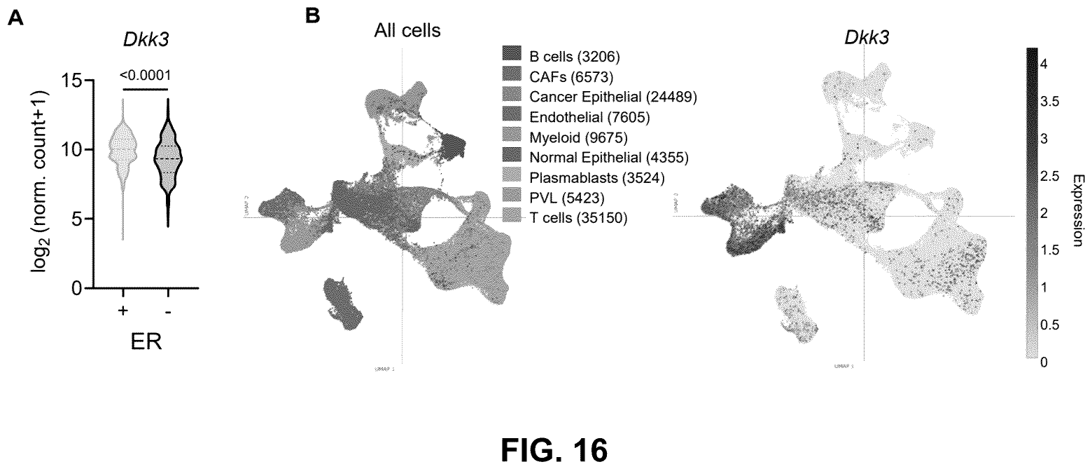

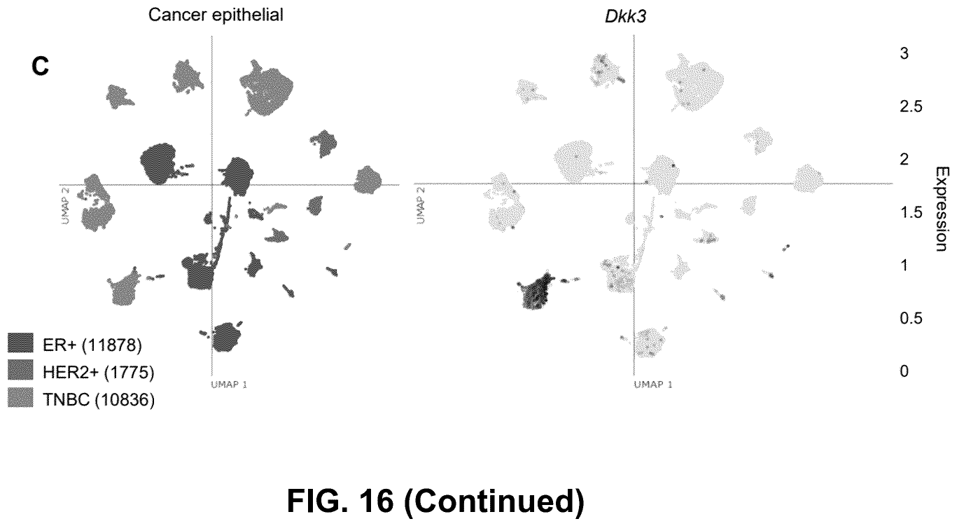

Interestingly, Dkk3 was inversely correlated with genes indicative of an anti-tumor immune response (e.g. Cd4, CD8a, Gzma, Gzmb, Ifng) in Basal BC across datasets, a pattern not observed with family members Dkk1 or Dkk2 (FIG. 7C; Table 2). Furthermore, Dkk3 was positively correlated with the ectonucleotidases Nt5e (CD73) and Entpd1 (CD39), which are highly expressed by Tregs and critically mediate peripheral tolerance(40), and negatively correlated with the proliferation marker Mki67 (FIG. 7C). Expression of Dkk3 was also higher in primary tumors of BC patients that presented with metastasis, even in ER+tumors which have higher Dkk3 at baseline (FIG. 7D, FIG. 16A). Single-cell RNA-seq analysis of human breast cancers revealed that Dkk3 is expressed by multiple cells within the breast microenvironment, including both normal and cancer epithelial cells, including TNBC cells(FIG. 16B,C). Thus, in human BC both the stroma or tumor cells themselves likely contribute to DKK3 in the microenvironment(41).

TABLE 2
Individual Pearson Correlation (2 tailed) and p values in reference to FIG. 7C.

| Pearson Correlation | | | p value | | |
|---|---|---|---|---|---|
| TCGA | KMplot | Metabric | Metabric | TCGA | KMplot |

Dkk1

| | | | | | | | |
|---|---|---|---|---|---|---|---|
| Cd4 | 0.0264 | −0.0233 | −0.1160 | Cd4 | 0.0943 | 0.7966 | 0.4717 |
| Cd8a | −0.0136 | −0.0569 | −0.0679 | Cd8a | 0.3288 | 0.8947 | 0.0793 |
| Cd8b | −0.0654 | −0.0429 | 0.0875 | Cd8b | 0.2079 | 0.5226 | 0.1858 |
| Gzma | 0.0416 | −0.0425 | −0.0330 | Gzma | 0.6351 | 0.6843 | 0.1900 |
| Gzmb | 0.05114 | −0.0375 | −0.0736 | Gzmb | 0.2897 | 0.6170 | 0.2481 |
| Prf1 | 0.0677 | −0.0565 | −0.0919 | Prf1 | 0.1855 | 0.5078 | 0.0815 |
| Ifng | 0.0315 | −0.0369 | −0.0957 | Ifng | 0.1680 | 0.7580 | 0.2303 |
| Tnf | 0.1736 | 0.0911 | 0.0347 | Tnf | 0.6179 | 0.0873 | 0.0049 |
| Entpd1 | 0.1020 | −0.0460 | 0.0364 | Entpd1 | 0.6009 | 0.3175 | 0.1560 |
| Nt5e | −0.0293 | −0.0541 | 0.0199 | Nt5e | 0.7754 | 0.7745 | 0.1225 |
| Mki57 | 0.0366 | 0.1186 | −0.1370 | Mki67 | 0.0479 | 0.7203 | 0.0002 |

Dkk2

| | | | | | | | |
|---|---|---|---|---|---|---|---|
| Cd4 | 0.4359 | 0.2434 | 0.1640 | Cd4 | 0.0176 | 0.0000 | <1E−04 |
| Cd8a | 0.3497 | 0.1844 | 0.1954 | Cd8a | 0.0046 | 0.0004 | <1E−04 |
| Cd8b | 0.1597 | 0.1516 | 0.0406 | Cd8b | 0.5594 | 0.1162 | <1E−04 |
| Gzma | 0.3253 | 0.1466 | 0.1696 | Gzma | 0.0141 | 0.0011 | <1E−04 |
| Gzmb | 0.2383 | 0.1155 | 0.1921 | Gzmb | 0.0053 | 0.0182 | 0.0004 |
| Prf1 | 0.2595 | 0.0993 | 0.1281 | Prf1 | 0.0646 | 0.0099 | 0.0021 |
| Ifng | 0.1952 | 0.0986 | 0.1806 | Ifng | 0.0089 | 0.0541 | 0.0023 |
| Tnf | 0.0421 | 0.0413 | −0.0340 | Tnf | 0.6253 | 0.6805 | 0.2022 |
| Entpd1 | 0.4891 | 0.2942 | −0.0080 | Entpd1 | 0.9085 | 0.0000 | <1E−04 |
| Nt5e | 0.1788 | 0.1237 | 0.0766 | Nt5e | 0.2704 | 0.0782 | 0.0001 |
| Mki67 | −0.1539 | −0.1074 | 0.0005 | Mki67 | 0.9944 | 0.1303 | 0.0009 |

Dkk3

| | | | | | | | |
|---|---|---|---|---|---|---|---|
| Cd4 | −0.0082 | 0.0017 | −0.2566 | Cd4 | 0.0002 | 0.9359 | 0.9581 |
| Cd8a | −0.1066 | −0.1632 | −0.2247 | Cd8a | 0.0011 | 0.2961 | <1E−04 |
| Cd8b | −0.0662 | −0.0433 | −0.1396 | Cd8b | 0.0438 | 0.5174 | 0.1817 |
| Gzma | −0.1059 | −0.0570 | −0.2086 | Gzma | 0.0024 | 0.2992 | 0.0784 |
| Gzmb | −0.2505 | −0.0585 | −0.3222 | Gzmb | <0.0001 | 0.0129 | 0.0711 |
| Prf1 | −0.1250 | −0.1009 | −0.3026 | Prf1 | <0.0001 | 0.2203 | 0.0018 |
| Ifng | −0.2758 | −0.2795 | −0.2937 | Ifng | <0.0001 | 0.0060 | <1E−04 |
| Tnf | −0.0804 | −0.1813 | −0.2160 | Tnf | 0.0017 | 0.4313 | <1E−04 |
| Entpd1 | 0.1883 | 0.1701 | 0.2799 | Entpd1 | <0.0001 | 0.0634 | <1E−04 |
| Nt5e | 0.5004 | 0.2883 | 0.3305 | Nt5e | <0.0001 | 0.0000 | <1E−04 |
| Mki67 | −0.1998 | −0.1454 | −0.4799 | Mki67 | <0.0001 | 0.0486 | <1E−04 |

### DKK3 Promotes Immune Evasion in Rapidly Progressing Models of BC

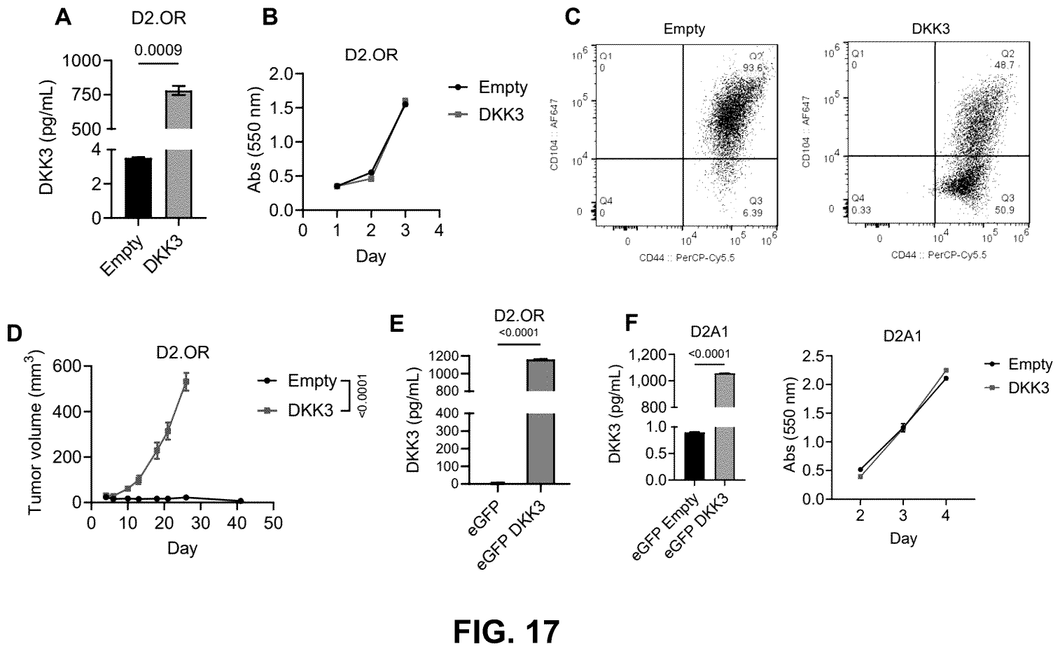

To directly assess the role of DKK3 in suppressing anti-tumor immunity, we utilized lentiviral vectors to stably overexpress DKK3 in D2.OR cells, which we found to be highly sensitive to adaptive immune responses (FIG. 17A; FIG. 1G). While DKK3 expression yielded no significant difference in cellular proliferation in vitro, it did elicit a CD44/CD104 profile similar to D2.1 cells or residual D2.OR tumors (FIG. 17B, C). Upon MFP implantation in immune competent hosts, DKK3 expression provided a robust engraftment and survival advantage compared to control empty vector D2.OR tumors, which were rejected after stable integration of the puromycin xenoantigen (FIG. 17D).

Prior reports suggest that DKK3 enables MHC-I mismatched transplantation of embryoid bodies(42), therefore to test the possibility that DKK3 protected against foreign antigen-specific immunity, a vector containing eGFP in addition to DKK3 was lentivirally transduced into D2.OR cells which were subsequently transplanted into the MFP of BALB/c mice. As with puromycin expressing D2.OR cells, we found that D2.OR cells expressing eGFP alone were completely rejected while parental D2.OR cells formed tumors (FIG. 7E). However, D2.OR cells expressing both eGFP and DKK3 successfully engrafted, although at a diminished rate compared to parental cells (FIG. 7E). Moreover, examination of infiltrating T cells revealed an increase in Tregs compared to parental tumors (FIG. 7F) and a systemic decrease in effector CD8+cells in the spleen (FIG. 7G). Together, these data suggest that DKK3 enables antigen-specific protection in otherwise immune-sensitive tumors and supports the role for DKK3 in Treg generation.

Congruent results were observed when expressing DKK3 in highly proliferative D2A1 cells (FIG. 17F). We found that DKK3-expressing D2A1 cells grew equivalently whether implanted into the MFP of Balb/c or SCID beige mice whereas control tumors were substantially delayed in Balb/c animals (FIG. 17G), and ELISA of serum at time of euthanasia confirmed that DKK3 expression was maintained (FIG. 17H). By the end of the experiment, effector CD8+T cells were significantly reduced in the tumor (FIG. 17I) and large alterations in splenic T cells were detected (FIG. 17J), with T cells were shifted towards a naïve (CD44−CD62l+) phenotype. Less anti-eGFP antibodies were also present in the serum of mice bearing DKK3-expressing D2A1 tumors when accounting for tumor volume (FIG. 17K). In sum, these in vivo data reveal that DKK3 can restrict adaptive anti-tumor immunity during multiple stages of tumor progression.

### Discussion

Tumor dormancy and re-emergence depend on both tumor-intrinsic and -extrinsic mechanisms that are slowly being uncovered. Our study does not address how tumors enter and exit dormancy (although others have recently shed light on dormancy in D2 cells(7)), however we describe an avenue by which intrinsically dormant and tumor populations evade adaptive immunity to eventually form progressive disease. This is achieved via early initiation of a CD4+/Treg skewed T cell response, and we provide evidence that the tumor-derived Wnt modulator DKK3 is critical to this process. We also demonstrate that DKK3 is essential for tumor survival during the latent phase and generally protects tumors from antigen-specific CD8+T cell immunity. Finally, our analysis suggests that DKK3 is a relevant target in BC, particularly in TNBC and Luminal B tumors.

An important determinant of recurrence is the duration that CD8+T cells maintain in their cytotoxic dominance against proliferative tumor cells (43, 44). Evidence suggests that CD39+PD-1+CD8+T cells represent a locally-induced, tumor-reactive population that can also prevent metastatic outgrowth in BC and others (10, 45). However, our studies revealed that dormant tumor cells prevent generation of this CD8+T cell population compared to more proliferative tumors.

We demonstrate that activated tumor antigen-specific CD8+T cells were unable to eliminate these latent tumors, but that targeting Tregs specifically allowed the generation of more effective anti-tumor responses, suggesting a primacy of Tregs in protecting dormant cancer cells.

The degree to which dormant tumors are targetable by CD8+T cells in general also remains elusive. It is commonly understood that MHC-I downregulation in dormant/disseminated tumor cells can promote immune escape (17, 46, 47), although our data suggest the importance of microenvironmental avenues of immune evasion as opposed to being “invisible” to adaptive immunity. A growing body of literature indicates that MHC-I/antigen presentation is highly dynamic (43, 48), supportive of other studies that have found no differences in antigen presentation pathway genes or MHC-I expression in dormant BC cells (20). Collectively, these studies suggest that, although they are in fact detectable by CD8+T cells, many dormant BC tumor cells rely on other means to escape elimination. While Tregs are generally important for tumor immune evasion (49), a definitive role in protecting dormant tumor cells remains elusive (50). Clinical studies suggest that Tregs are specifically associated with late relapse (>5 years) and poor metastatic survival in BC (51, 52).

We found direct evidence that tumor-derived DKK3 was able to induce Treg differentiation in vitro which ultimately resulted in decreased CD8+T cell function. While others have reported direct effects of DKK3 on CD8+cells (42, 53, 54), we found the effect was most prominent on CD4+T cells/Tregs. Recently, DKK3 was described to regulate downstream Wnt/β-catenin genes Tef7, which encodes T cell factor-1 (TCF-1), and Lef1 (encoding Lymphoid Enhancer Binding Factor 1), in CD4+cells to coordinate IFNγ secretion by a yet to be determined receptor (55). Canonical Wnt signaling also inhibits Treg activity(56), therefore suppression of Wnt by tumor-derived DKK3 may mediate CD4 differentiation towards Tregs. Additionally, other studies support a role for DKK3 in concert with other factors in the extracellular milieu, such as TGF-β (57-60). The TGF-β pathway is especially relevant for Treg function(61), and because D2.1 cells also expressed more Tgfb1 we speculate that this may be an important connection to delineate the contextual function of DKK3 on T cells in addition to Wnt.

Currently the role of DKK3 in cancer is poorly understood with both inhibitory and beneficial functions being reported in tumors (41, 62-68). In our hands DKK3 did not directly alter tumor cell proliferation, but instead showed tumor-promoting effects when in contact with the immune system. This is consistent with recent studies in pancreatic tumors that demonstrated increased T cell accumulation in DKK3 KO mice and others showing greater tumor rejection with enhanced CD8+T cell infiltration into tumors when mesenchymal stem cells were DKK3 deficient (54, 69). More generally, DKK3 was identified as necessary for generating antigen-specific, tolerant CD8+T cells in vivo and could suppress T cell proliferation in vitro (53). In an autoimmune context, DKK3 produced locally by stromal cells in the skin was found to reduce self-antigen induced experimental autoimmune encephalomyelitis (EAE) symptoms in a CD4+T cell-dependent fashion (42, 70). As such, our results further elucidate an ability of DKK3 to restrain adaptive immunity in normal and pathological conditions through the induction of Tregs, although the exact molecular drivers of this process will need further clarification.

Along with other secreted factors, other cell types in specific niches may play a critical role in DKK3 expression and function. Our results were obtained in an orthotopic site (i.e. the MFP) with tumor-derived cytokines. As mentioned previously, within the human mammary gland Dkk3 is one of the most upregulated genes in CD44+CD24−(stem) vs CD44−CD24+(differentiated) cells(38), which mimics the phenotype of D2.1 vs D2A1/D2.OR cells. However, other cells within the local tumor microenvironment and certain disseminated niches may also be important contributors of DKK3. Interestingly, DKK3 is commonly expressed by stromal cells in “immune privileged” and stem cell niches in the brain, eye, pancreas, liver, and bone marrow (39, 71-76).

The trophectoderm, which induces immune tolerance at the maternal-fetal interface, in part by recruiting and/or inducing Tregs, is also enriched for DKK3 expression (77-79). Thus, DKK3 may be a component of larger mechanisms that maintain stem cell niches and prevent aberrant immune activation against these critical populations, which we speculate is driven by the plastic E/M phenotype (12) and the fact that DKK3 can prevent MHC-I mismatched embryonic body rejection (42) supports this notion. Currently, immune checkpoint blockade (ICB) remains largely unsuccessful in hormone receptor positive BC (which are most likely to go dormant) due to low T cell infiltration and an immunosuppressive stroma (80). Although pancreatic cancer is similarly resistant to ICB, combined anti-CTLA4+anti-DKK3 antibodies showed synergistic anti-tumor responses in a pancreatic cancer model (54). Although ICB has been approved for certain TNBCs, great interest also remains in combinatorial therapies that target alternative pathways to boost ICB response (81). Therefore, targeting DKK3 alongside other immunotherapeutic strategies may help break alternative suppressive barriers to eliminate dormant, residual tumor cells and more aggressive cells alike.

### Methods

### Mice

SCID-beige (C.B-Igh-1b/GbmsTac-Prkdcscid-Lystbg N7; model CBSCBG) and BALB/c (BALB/cAnNTac; model BALB) were purchased from Taconic Biosciences. Jedi mice (Ptprca TcrbLn1Bdb TcraLn1Bdb H2d/J; strain 028062) were purchased from Jackson Laboratories and subsequently crossed to Rag1−/−(B6.129S7-Rag1tm1Mom/J; strain 002216) from Jackson Laboratories. FoxP3-DTR mice (B6.129(Cg)-Foxp3tm3(DTR/GFP)Ayr/J; strain 016958) were purchased from Jackson Labs and female FoxP3-DTR mice were crossed with male BALB/c to generate F1 animals for implantation. Genotyping and genetic monitoring for all strains were routinely performed by Transnetyx. Female mice between 8-12 weeks of age were used for tumor studies. Cell lines D2A1, D2.OR, and D2.1 cells were generously provided by Dr. Ann Chambers (London Health Sciences Centre, London, Ontario). Cell lines were confirmed mycoplasma negative via MycoStrip (InvivoGen) and were maintained in low-glucose DMEM (Gibco) containing 10% FBS and penicillin(10 U/mL)/streptomycin(10 μg/mL) at 37° C. and 5% CO2. For a summary of D2 cell phenotypes see Table 3. Plasmids The LeGO-G lentiviral plasmid (Addgene #27347) was used to generate eGFP+D2A1, D2.OR, and D2.1 cells. shRNA lentiviral vectors (pLKO.1) targeting Dkk3 were obtained from Sigma (TRCN0000071752; target sequence TCTGTGACAACCAGAGGGATT; SEQ ID NO: 1). Gene targeting of Dkk3 by CRISPR/Cas9 was accomplished via pLentiCRISPRv2 (Addgene # 52961) and sgRNA sequences for Dkk3 were obtained from the GeCKO v2 CRISPR library provided by Feng Zhang (Addgene #1000000052)(82). Inducible Dkk3 vectors were modified from previously published vectors(83).

### Lentiviral Production and Infection

All lentiviral vectors were produced in 293T cells using second-generation packaging plasmids and previously described techniques and viral stocks were concentrated by ultracentrifugation. Viral stocks were added to medium containing 8 μg/mL polybrene and cells were incubated for 48 hours before sub-culturing. Transduced cells were selected via puromycin (shRNA knockdown, CRISPR knockout) or sorting for eGFP+cells. Single cell clones were generated by limited dilution in 96 well-plates and subsequent culture of wells containing a single colony.

### Orthotopic Implantation

Tumor cells in sterile PBS were implanted into the fourth inguinal mammary fat pads under general anesthesia. Tumors were monitored by caliper and volume was calculated using the formula volume=(length×width2)÷2.

TABLE 3
Overall phenotype of D2 cells as described herein and by others.

| Cell | | | | | Immune | | |
|---|---|---|---|---|---|---|---|
| Line | Derived from | Strain | Syngeneic | Dormant? | Sensitive? | EMT Status | ER+ |
| D2A1 | D2HAN | Balb/c | Yes | No | Medium | mesenchymal | No |
| D2.OR | (D2-type | Balb/c | Yes | Yes | High | Hybrid | Yes |
| | hyperplastic | | | (lungs¹⁶) | | | |
| D2.1 | alveolar | Balb/c | Yes | Yes | Low | Hybrid | Yes |
| | nodules)¹⁴ | | | (fat pad, | | | |
| | | | | lungs^{15, 16}) | | | |

### Antibody/DT Treatment

Antibodies against CTLA4 (clone 9D9, BioXCell) were administered intraperitoneally (IP) biweekly at 200 μg/mouse. Diphtheria toxin (Cayman Chemical) was given IP at 25 ng/g every 4 days with a total of 5 doses. T cell activation and adoptive transfer 6 well plates were pre-coated overnight with 0.5 μg/mL anti-CD38 (BioXCell, clone 145-2C11) and 5 μg/mL anti-CD28 (BioXCell, clone 37.51) in PBS (5 mL/well) at 4° C. Jedi spleens were crushed through a 70 μm filter, red blood cells were lysed with ACK buffer, and cells were plated at 5×10⁶ cells/mL and 5 mL/well in RPMI 1640 medium containing 10% FBS, L-glutamine (2 mM), penicillin (10 U/mL)/streptomycin (10 μg/mL), 2-mercaptoethanol (50 μM), and 1×Insulin-Transferrin-Selenium (ITS-G; Gibco) and cultured overnight at 37° C. and 5% CO2. The following day, recombinant mouse IL-2 (30 U/mL; BioLegend) and IL-7 (0.5 ng/ml; BioLegend) were added to the culture medium and cells were incubated at 37° C. for 24 hours. Cells were subsequently sub-cultured at 10⁶ cells/mL in fresh medium containing IL-2 and IL-7 and incubated an additional 24 hours. Expanded T cells were washed 2×, resuspended in sterile PBS at 10⁶ cells/50 μL, and injected IV into mice under general anesthesia (10⁶ cells/mouse).

### Killing Assays

D2A1 or D2.1 target cells expressing eGFP-Luc were plated at 2,000 cells/well in a 96 well plate and allowed to attach overnight at 37° C. The following day, serial dilutions of effector Jedi T cells (activated as for adoptive transfer) were added to each well in quadruplicate and plates were incubated overnight at 37° C. Cells were lysed with a Triton-x 100 buffer and luciferase activity was measured using a Veritas microplate luminometer (Turner Biosystems). The fraction of remaining cells in each well was normalized to the average signal of control wells that did not receive Jedi cells.

Conditioned Medium (CM) Harvest Tumor cells were seeded in complete medium at 5×10³ cells/mm2 in a 10 cm dish and allowed to attach before changing medium to low-glucose DMEM containing 1% FBS. Cells were cultured for 48 hours at which point conditioned medium was collected, centrifuged to remove debris, passed through a 0.45 μm filter, and stored at 4° C. until use.

### T Cell Activation Proliferation Assays

CD4/CD8 T cell isolation: Splenocytes were resuspended in EasySep buffer (Stemcell Technologies) at 10⁶ cells/mL and CD4 cells were isolated with EasySep Mouse CD4+T Cell Isolation Kit (Stemcell Technologies) or CD8+cells were isolated using the Easy Sep Mouse CD8+T Cell Isolation Kit (Stemcell Technologies) according to the manufacturer's protocol. CellTrace staining: Single cells (whole splenocytes or CD8+cells only) were resuspended in PBS at 10⁶ cells/mL. Cells were stained with 1 μL/mL (after reconstitution in 20 μL DMSO) CellTrace Far Red (Invitrogen) for 20 minutes at 37° C. with gentle mixing every 5 minutes. Cell suspension was diluted 5×with complete medium and incubated at room temperature for 5 minutes and washed 1×with medium. CM culture: For experiments with whole splenocytes, CellTrace stained Jedi splenocytes were plated in a 1:1 mixture of D2 CM:fresh medium containing anti-CD28 antibodies (2 μg/mL), eGFP₂₀₀₋₂₀₈ peptide (1 μg/mL), and 2-mercaptoethanol (50 μM) at 7.2×10⁵ cells/well in a 24 well plate. Cells were cultured at 37° C. for 72 hours and floating/weakly attached cells were stained for Live/Dead, CD45, CD4, CD8β, and FoxP3. For experiments with CD8+cells alone, 4.1×10⁵ CellTrace stained Jedi Rag1−/−CD8+cells/well and 4.1×10⁵ splenocytes from SCID-beige mice (serving as APCs expressing H-2Kd) from were plated with eGFP peptides as before. For experiments with CD8+, CD4, and APCs, 2.4×10⁵ CD4+cells (from BALB/c), 2.4×10⁵ CellTrace stained Jedi Rag1−/−CD8+cells, and 2.4×10⁵ SCID beige splenocytes were plated per well with eGFP peptides as before.

### Immunohistochemistry (IHC)/Immunofluorescence (IF)

Formalin-fixed, paraffin-embedded tissue sections were deparaffinized with xylene and rehydrated through graded concentrations of ethanol and distilled water. Epitope retrieval was performed in a Retriever 2100 (Aptum Biologics) with R-Buffer A (Electron Microscopy Sciences). IHC: Endogenous peroxidases/phosphatases were quenched with BLOXALL blocking solution (Vector) and tissues were blocked with Animal-Free Blocker R.T.U. (Vector). Sections were probed with primary antibodies (for a complete list see Table 4) overnight at 4° C., washed with PBS, and incubated with the appropriate ImmPRESS polymer detection reagent (Vector) for 30 minutes at room temperature. Visualization was performed by incubation with 3,3′-diaminobenzidine (DAB) (Vector), ImmPACT Vector Red (Vector), or a Green HRP staining kit (Novus), For triple IHC, a second round of retrieval was performed with R-Buffer A after developing the first round of HRP and AP stains. Tissues were counterstained with Gill No.3 Hematoxylin (Sigma), cover-slipped, and imaged on an Olympus IX73 inverted microscope with a 40×objective. Infiltrated T cells were enumerated on 5 random fields of view per sample using ImageJ software. IF: Tissues were blocked with Animal-Free Blocker R.T.U. and incubated in primary antibodies overnight at 4° C. Tissues were washed with PBS, and secondary staining was performed for 1 hour in the dark at room temperature with the appropriate fluorophore-conjugated antibody. Tissues were counterstained with DAPI and cover-slipped with VECTASHIELD Vibrance (Vector) mounting media. Whole-slide images were collected using a Zeiss LSM880 confocal microscope. Ki67 staining analysis was performed using CellProfiler software. Single nuclei were segmented using the DAPI channel and the eGFP channel was used to create a binary mask to delineate between tumor and stroma. The number of Ki67+nuclei within the eGFP mask was quantified and represented as a percent of the total nuclei within the eGFP mask.

TABLE 4
Antibodies used for staining.

| Target | Conjugate | Clone | Catalog # | Vendor | Use |
|---|---|---|---|---|---|
| CD104 | APC | 346-11A | 123611 | BioLegend | Flow cytometry |
| CD104 | AF647 | 346-11A | 123607 | BioLegend | Flow cytometry |
| CD11b | PerCP Cy5.5 | M1/70 | 101227 | BioLegend | Flow cytometry |
| CD11b | AF700 | M1/70 | 101222 | BioLegend | Flow cytometry |
| CD24 | PE Dazzle 594 | M1/69 | 101837 | BioLegend | Flow cytometry |
| CD39 | PE Dazzle 594 | Duha59 | 143811 | BioLegend | Flow cytometry |
| CD4 | Unconjugated | D7D2Z | 25229 | Cell Signaling | IHC |
| CD4 | PE Dazzle 594 | RM4-5 | 100566 | BioLegend | Flow cytometry |
| CD4 | BV421 | RM4-5 | 100543 | BioLegend | Flow cytometry |
| CD44 | PerCP Cy5.5 | IM7 | 103032 | BioLegend | Flow cytometry |
| CD45 | BV605 | 30-F11 | 103140 | BioLegend | Flow cytometry |
| CD47 | PE | miap301 | 127507 | BioLegend | Flow cytometry |
| CD62L | PE | MEL-14 | 104407 | BioLegend | Flow cytometry |
| CD8b | APC Cy7 | YTS156.7.7 | 126620 | BioLegend | Flow cytometry |
| CD8b | PE Cy7 | YTS156.7.7 | 126616 | BioLegend | Flow cytometry |
| CD8b | BV605 | H35-17.2 | 740387 | BD Biosciences | Flow cytometry |
| CD8α | Unconjugated | D4W2Z | 98941 | Cell Signaling | IHC |
| GFP | Unconjugated | Polyclonal | GFP-1020 | Aves Labs | IF |
| FoxP3 | Unconjugated | D6O8R | 12653S | Cell Signaling | IHC |
| FoxP3 | AF488 | MF-14 | 126406 | BioLegend | Flow cytometry |
| FoxP3 | Unconjugated | FJK-16s | 14-5773-82 | Invitrogen | IHC |
| Ki-67 | Unconjugated | Polyclonal | NB110-89717SS | Novus Biologicals | IF |
| MHC I H2Kd/H2Dd | PE | 34-1-2S | 114713 | BioLegend | Flow cytometry |
| PD-1 (CD279) | BV421 | 29F.1A12 | 135218 | BioLegend | Flow cytometry |
| PD-L1 (CD274) | PE | 10F.9G2 | 124307 | BioLegend | Flow cytometry |

### Tumor Cell Proliferation Assays

Cells were plated at 4×10³ cells/well in a 96 well plate and cultured at 37° C. Cells were incubated in medium containing 0.5 mg/mL 3-[4,5-dimethylthiazol-2-yl]-2,5-diphenyltetrazolium bromide (MTT) for 4 hours at 37° C., gently washed, and crystalline formazan was dissolved in DMSO. Plates were read at 550 nm with a Bio-Rad 680 microplate reader.

### Quantitative Real-Time PCR

RNA was isolated using Qiagen RNAeasy kits and reverse transcribed using iScript cDNA Synthesis Kit (Bio Rad) before performing quantitative PCR using Sso Advanced Universal SYBR Green Supermix (Bio Rad). Values were determined using the AACT method with actin as internal control.

### Flow Cytometry

Tumors were digested in serum-free medium using collagenase (1 mg/mL), DNAse (20 U/mL), and hyaluronidase (100 μg/mL) for 90 minutes at 37° C. Spleens were mechanically dissociated as before. Samples were washed with serum-containing medium, red blood cells were lysed with ACK buffer, and passed through a 40-μm cell strainer to generate single cell suspensions. For each condition 2×10⁶ cells were stained at 4° C. with Fc Block (CD16/32 clone 93; BioLegend), Live/Dead Fixable Dye (Invitrogen) and a combination of directly conjugated antibodies (for a complete list see Table 4). Cells were fixed in 1% paraformaldehyde and analyzed on a BD LSR or Cytek Northern Lights flow cytometer. Compensation was performed using single color stained splenocytes or BD CompBeads in FlowJo software (v10) which was also used for downstream analysis. After live gating, T cells were identified as CD45+CD11b−CD4+/CD8+, CD4 cells were identified as CD45+CD11b−CD4+and divided into Tregs CD45+CD11b−CD4+CD8−Foxp3+or T helper CD45+CD11b−CD4+CD8−Foxp3−, and CD8 cells were identified by CD45+CD11b−CD8+(FIG. 12A).

### Quantitative ELISA

Mouse IFNγ in T cell cultures was detected using the ELISA MAX Set from BioLegend according to the manufacturer's protocol. Anti-mouse DKK3 ELISA kits were purchased from RayBiotech and serum DKK3 was detected according to the standard protocol. RNA-seq Individual tumors were snap frozen and stored in RNAlater-ICE (Invitrogen) until RNA extraction. Total RNA was extracted using PureLink RNA mini Kits (Invitrogen) and DNA was removed via DNA-free DNA Removal kit (Invitrogen). RNA quality and concentration was determined using an Agilent 2100 Bioanalyzer. Whole transcriptome sequencing of cell lines and tumors was performed by Novogene on an Illumina NovaSeq 6000. Read alignment, quality control, differential expression analysis, and pathway analysis was performed by Novogene using the standard pipeline or GENAVi (84). CIBERSORT analysis was performed by TIMER2.0 using the immune estimation function. Gene-set enrichment analysis (GSEA; version 4.2.3) was performed on DESeq2 normalized counts using the gene_set permutation type with Mammary stem/progenitor signatures(28) accessed via the MsigDB and previously published late recurrence or late distant metastasis signatures(30).

### Anti-GFP ELISA

Immulon 4 HBX (Thermo Scientific) plates were coated with 1 μg/mL recombinant A. victoria GFP (Abcam) at 4° C. The following day plates were washed with PBS+0.05% Tween 20 and blocked with PBS+1% BSA (Sigma) for 1 hour at 37° C. A serial dilution (in 1% BSA/PBS) of serum was added in duplicate for 2 hours at room temperature followed by anti-mouse IgG streptavidin-HRP conjugated antibody (1:2000 in 1% BSA/PBS; Cell Signaling Technology) for 1 hour at 37° C. Plates were developed with TMB substrate (BioLegend), stopped with 0.18 M H2SO4, and read at 450 nm on a Bio-Rad 680 microplate reader.

### Clinical Analysis

Survival and expression correlation analysis of select genes was performed using the Kaplan-Meier Plotter tool (Breast cancer, mRNA gene chip). Expression levels of Dkk3 (Affymetrix ID 214247_s_at) were split by upper and lower quartiles to compare relapse free survival (RFS) by PAM50 subtype. Overall survival (OS) data, PAM50 classification, and RNAseq log2 normalized counts for the TCGA-BRCA dataset was accessed via UCSC Xena Browser and stratified based upon upper and lower Dkk3 quartiles for survival. METABRIC and the Metastatic Breast Cancer Project mRNA expression data and clinical characteristics were accessed via cBioPortal.

### Statistical Analysis

No statistical methods were used to pre-determine animal numbers and when appropriate animals were randomly assigned to treatment groups using the RANDBETWEEN function in Excel. Data were visualized and analyzed using GraphPad Prism v9 with each point representing a single mouse for in vivo experiments or technical replicates for in vitro experiments. In vitro experiments were performed at least twice with similar results and in vivo experiments were repeated with similar results or validated with complimentary experiments. Details of statistical analyses can be found in the figure legends and p values are displayed within the figures. P values≤0.05 were considered significant. All animal studies were performed in accordance with Duke IACUC approval (protocol A043-23-02) and supervised/housed by the Division of Laboratory Animal Resources (DLAR). Data availability The tumor and cell line bulk RNA-Seq data has been submitted to the NCBI's Gene Expression Omnibus (GEO) database (GSE240021). Individual data values for the main figures and supplement can be found in the “Supporting data values” file.

### References

1. Harbeck N, Penault-Llorca F, Cortes J, Gnant M, Houssami N, Poortmans P, et al. Breast cancer. Nature Reviews Disease Primers. 2019;5(1):66.

2. Janni W, Vogl F D, Wiedswang G, Synnestvedt M, Fehm T, Juckstock J, et al. Persistence of disseminated tumor cells in the bone marrow of breast cancer patients predicts increased risk for relapse—a European pooled analysis. Clinical cancer research an official journal of the American Association for Cancer Research. 2011; 17(9):2967-74
3. Wu X, Baig A, Kasymjanova G, Kafi K, Holcroft C, Mekouar H, et al. Pattern of Local Recurrence and Distant Metastasis in Breast Cancer By Molecular Subtype. Cureus. 2016;8(12):e924.
4. Dent R, Trudeau M, Pritchard K I, Hanna W M, Kahn H K, Sawka C A, et al. Triple-negative breast cancer: clinical features and patterns of recurrence. Clinical cancer research: an official journal of the American Association for Cancer Research. 2007; 13(15 Pt 1):4429-34.
5. Bushnell G G, Deshmukh A P, den Hollander P, Luo M, Soundararajan R, Jia D, et al. Breast cancer dormancy: need for clinically relevant models to address current gaps in knowledge. NPJ Breast Cancer. 2021;7(1):66.
6. Phan T G, and Croucher P I. The dormant cancer cell life cycle. Nature Reviews Cancer. 2020;20(7):398-411.
7. Di Martino J S, Nobre A R, Mondal C, Taha I, Farias E F, Fertig E J, et al. A tumor-derived type III collagen-rich ECM niche regulates tumor cell dormancy. Nature Cancer. 2022;3(1):90-107.
8. Borriello L, Coste A, Traub B, Sharma V P, Karagiannis G S, Lin Y, et al. Primary tumor associated macrophages activate programs of invasion and dormancy in disseminating tumor cells. Nature Communications. 2022; 13(1):626.
9. Dunn G P, Old L J, and Schreiber R D. The immunobiology of cancer immunosurveillance and immunoediting. Immunity. 2004;21(2): 137-48.
10. Tallon de Lara P, Castanon H, Vermeer M, Nunez N, Silina K, Sobottka B, et al. CD39(+)PD-1(+)CD8(+) T cells mediate metastatic dormancy in breast cancer. Nat Commun. 2021; 12(1):769.
11. Piranlioglu R, Lee E, Ouzounova M, Bollag R J, Vinyard A H, Arbab A S, et al. Primary tumor-induced immunity eradicates disseminated tumor cells in syngeneic mouse model. Nature Communications. 2019;10(1):1430.
12. Crump L S, Kines K T, Richer J K, and Lyons T R. Breast cancers co-opt normal mechanisms of tolerance to promote immune evasion and metastasis. American Journal of Physiology-Cell Physiology. 2022;323(5):C1475-C95.
13. Morris V L, Tuck A B, Wilson S M, Percy D, and Chambers A F. Tumor progression and metastasis in murine D2 hyperplastic alveolar nodule mammary tumor cell lines. Clinical & experimental metastasis. 1993;11(1):103-12.
14. Rak J W, McEachern D, and Miller F R. Sequential alteration of peanut agglutinin binding-glycoprotein expression during progression of murine mammary neoplasia. Br J Cancer. 1992;65(5):641-8.
15. Shibue T, Brooks M W, and Weinberg R A. An integrin-linked machinery of cytoskeletal regulation that enables experimental tumor initiation and metastatic colonization. Cancer cell. 2013;24(4):481-98.
16. Shibue T, and Weinberg R A. Integrin betal-focal adhesion kinase signaling directs the proliferation of metastatic cancer cells disseminated in the lungs. Proc Natl Acad Sci U S A. 2009; 106(25):10290-5.677
17. Aguirre-Ghiso J A. How dormant cancer persists and reawakens. Science. 2018;361(6409):1314-5.
18. Zhang H, Lu H, Xiang L, Bullen J W, Zhang C, Samanta D, et al. HIF-1 regulates CD47 expression in breast cancer cells to promote evasion of phagocytosis and maintenance of cancer stem cells. Proceedings of the National Academy of Sciences.2015; 112(45):E6215-E23.
19. Grzelak C A, Goddard E T, Lederer E E, Rajaram K, Dai J, Shor R E, et al. Elimination of fluorescent protein immunogenicity permits modeling of metastasis in immune-competent settings. Cancer cell. 2022;40(1):1-2.
20. Baldominos P, Barbera-Mourelle A, Barreiro O, Huang Y, Wight A, Cho J-W, et al. Quiescent cancer cells resist T cell attack by forming an immunosuppressive niche. Cell. 2022; 185(10):1694-708.e19.
21. Rae J M, Johnson M D, Scheys J O, Cordero K E, Larios J M, and Lippman M E. GREB 1 is a critical regulator of hormone dependent breast cancer growth. Breast Cancer Res Treat. 2005;92(2): 141-9.
22. Sheridan C, Kishimoto H, Fuchs R K, Mehrotra S, Bhat-Nakshatri P, Turner C H, et al. CD44+/CD24−breast cancer cells exhibit enhanced invasive properties: an early step necessary for metastasis. Breast Cancer Res. 2006;8(5):R59.
23. Li W, Ma H, Zhang J, Zhu L, Wang C, and Yang Y. Unraveling the roles of CD44/CD24 and ALDH1 as cancer stem cell markers in tumorigenesis and metastasis. Scientific Reports. 2017;7(1):13856.
24. Kröger C, Afeyan A, Mraz J, Eaton E N, Reinhardt F, Khodor Y L, et al. Acquisition of a hybrid E/M state is essential for tumorigenicity of basal breast cancer cells. Proceedings of the National Academy of Sciences. 2019; 116(15):7353-62.
25. Grosse-Wilde A, Fouquier d′Hérouël A, McIntosh E, Ertaylan G, Skupin A, Kuestner R E, et al. Stemness of the hybrid Epithelial/Mesenchymal State in Breast Cancer and Its Association with Poor Survival. PloS one. 2015; 10(5):e0126522.
26. Grasset E M, Dunworth M, Sharma G, Loth M, Tandurella J, Cimino-Mathews A, et al. Triple-negative breast cancer metastasis involves complex epithelial-mesenchymal transition dynamics and requires vimentin. Sci Transl Med. 2022; 14(656):eabn7571.
27. Mullins R D Z, Pal A, Barrett T F, Heft Neal M E, and Puram S V. Epithelial-Mesenchymal Plasticity in Tumor Immune Evasion. Cancer Research. 2022;82(13):2329-43.
28. Lim E, Wu D, Pal B, Bouras T, Asselin-Labat M L, Vaillant F, et al. Transcriptome analyses of mouse and human mammary cell subpopulations reveal multiple conserved genes and pathways. Breast Cancer Res. 2010; 12(2):R21.
29. Cheng Q, Chang J T, Gwin W R, Zhu J, Ambs S, Geradts J, et al. A signature of epithelial-mesenchymal plasticity and stromal activation in primary tumor modulates late recurrence in breast cancer independent of disease subtype. Breast Cancer Research. 715 2014;16(4):407.
30. Mittempergher L, Saghatchian M, Wolf D M, Michiels S, Canisius S, Dessen P, et al. A gene signature for late distant metastasis in breast cancer identifies a potential mechanism of late recurrences. Molecular Oncology. 2013;7(5):987-99.
31. Oki T, Nishimura K, Kitaura J, Togami K, Maehara A, Izawa K, et al. A novel cell-cycle-indicator, mVenus-p27K−, identifies quiescent cells and visualizes G0-G1 transition. Scientific Reports. 2014;4.
32. Hollern D P, Yuwanita I, and Andrechek E R. A mouse model with T58A mutations in Myc reduces the dependence on KRas mutations and has similarities to claudin-low human breast cancer. Oncogene. 2013;32(10):1296-304.
33. Hammerl D, Martens J W M, Timmermans M, Smid M, Trapman-Jansen A M, Foekens R, et al. Spatial immunophenotypes predict response to anti-PD1 treatment and capture distinct paths of T cell evasion in triple negative breast cancer. Nature Communications. 2021; 12(1):5668.
34. Agudo J, Ruzo A, Park E S, Sweeney R, Kana V, Wu M, et al. GFP-specific CD8 T cellsenable targeted cell depletion and visualization of T-cell interactions. Nature biotechnology. 2015;33(12):1287-92.
35. Jin D, Fan J, Wang L, Thompson L F, Liu A, Daniel B J, et al. CD73 on Tumor Cells Impairs Antitumor T-Cell Responses: A Novel Mechanism of Tumor-Induced Immune Suppression. Cancer Research. 2010;70(6):2245-55.
36. Selby M J, Engelhardt J J, Quigley M, Henning K A, Chen T, Srinivasan M, et al. Anti-CTLA-4 antibodies of IgG2a isotype enhance antitumor activity through reduction of intratumoral regulatory T cells. Cancer immunology research. 2013; 1(1):32-42.
37. Crosby E J, Wei J, Yang X Y, Lei G, Wang T, Liu C X, et al. Complimentary mechanisms of dual checkpoint blockade expand unique T-cell repertoires and activate adaptive anti-tumor immunity in triple-negative breast tumors. Oncoimmunology. 2018; 7(5):e1421891.
38. Colacino J A, Azizi E, Brooks M D, Harouaka R, Fouladdel S, McDermott S P, et al. Heterogeneity of Human Breast Stem and Progenitor Cells as Revealed by Transcriptional Profiling. Stem Cell Reports. 2018; 10(5):1596-609.
39. Helbling P M, Piñeiro-Yáñez E, Gerosa R, Boettcher S, Al-Shahrour F, Manz M G, et al. Global Transcriptomic Profiling of the Bone Marrow Stromal Microenvironment during Postnatal Development, Aging, and Inflammation. Cell Reports. 2019;29(10):3313-30.e4.
40. Allard B, Longhi M S, Robson S C, and Stagg J. The ectonucleotidases CD39 and CD73: Novel checkpoint inhibitor targets. Immunological Reviews. 2017;276(1):121-44. 750
41. Ferrari N, Ranftl R, Chicherova I, Slaven N D, Moeendarbary E, Farrugia A J, et al. Dickkopf-3 links HSF1 and YAP/TAZ signalling to control aggressive behaviours in cancer-associated fibroblasts. Nature Communications. 2019; 10(1):130.
42. Meister M, Papatriantafyllou M, Nordstrom V, Kumar V, Ludwig J, Lui K O, et al. Dickkopf-3, a tissue-derived modulator of local T-cell responses. Front Immunol. 2015;6:78.
43. Romero I, Garrido C, Algarra I, Collado A, Garrido F, and Garcia-Lora A M. T Lymphocytes Restrain Spontaneous Metastases in Permanent Dormancy. Cancer Research. 2014;74(7):1958-68.
44. Eyles J, Puaux A-L, Wang X, Toh B, Prakash C, Hong M, et al. Tumor cells disseminate early, but immunosurveillance limits metastatic outgrowth, in a mouse model of melanoma. The Journal of Clinical Investigation. 2010; 120(6):2030-9.
45. Lee Y J, Kim J Y, Jeon S H, Nam H, Jung J H, Jeon M, et al. CD39<sup>+</sup>tissue-resident memory CD8<sup>+</sup>T cells with a clonal overlap across compartments mediate antitumor immunity in breast cancer. Science Immunology. 2022; 7(74):eabn8390.
46. Pommier A, Anaparthy N, Memos N, Kelley Z L, Gouronnec A, Yan R, et al. Unresolved endoplasmic reticulum stress engenders immune-resistant, latent pancreatic cancer metastases. Science. 2018;360(6394):eaao4908.
47. Pantel K, Schlimok G, Kutter D, Schaller G, Genz T, Wiebecke B, et al. Frequent down-regulation of major histocompatibility class I antigen expression on individual micrometastatic carcinoma cells. Cancer Res. 1991;51(17):4712-5.
48. Chang C-L, Wu C-C, Hsu Y-T, and Hsu Y-C. Immune vulnerability of ovarian cancer stem-like cells due to low CD47 expression is protected by surrounding bulk tumor cells. Oncoimmunology. 2020;9(1):1803530.
49. Gonzalez-Navajas J M, Fan D D, Yang S, Yang F M, Lozano-Ruiz B, Shen L, et al. The Impact of Tregs on the Anticancer Immunity and the Efficacy of Immune Checkpoint Inhibitor Therapies. Front Immunol. 2021; 12:625783.
50. Manjili M H, and Butler S E. Role of Tregs in Cancer Dormancy or Recurrence. Immunol Invest. 2016;45(8):759-66.
51. Bates G J, Fox S B, Han C, Leek R D, Garcia J F, Harris A L, et al. Quantification of Regulatory T Cells Enables the Identification of High-Risk Breast Cancer Patients and Those at Risk of Late Relapse. Journal of Clinical Oncology. 2006;24(34):5373-80. 783
52. Stenström J, Hedenfalk I, and Hagerling C. Regulatory T lymphocyte infiltration in metastatic breast cancer—an independent prognostic factor that changes with tumor progression. Breast Cancer Research. 2021;23(1):27.
53. Papatriantafyllou M, Moldenhauer G, Ludwig J, Tafuri A, Garbi N, Hollmann G, et al. Dickkopf-3, an immune modulator in peripheral CD8 T-cell tolerance. Proc Natl Acad Sci U S A. 2012;109(5):1631-6.
54. Zhou L, Husted H, Moore T, Lu M, Deng D, Liu Y, et al. Suppression of stromal-derived Dickkopf-3 (DKK3) inhibits tumor progression and prolongs survival in pancreatic ductal adenocarcinoma. Sci Transl Med. 2018;10(464).
55. Chen X, Hu J, Wang Y, Lee Y, Zhao X, Lu H, et al. The FoxO4/DKK3 axis represses IFN-γ expression by Th1 cells and limits antimicrobial immunity. The Journal of Clinical Investigation. 2022;132(18).
56. van Loosdregt J, Fleskens V, Tiemessen Machteld M, Mokry M, van Boxtel R, Meerding J, et al. Canonical Wnt Signaling Negatively Modulates Regulatory T Cell Function. Immunity. 2013;39(2):298-310.
57. Snelling S J B, Davidson R K, Swingler T E, Le L T T, Barter M J, Culley K L, et al. Dickkopf-3 is upregulated in osteoarthritis and has a chondroprotective role. Osteoarthritis and Cartilage. 2016;24(5):883-91.
58. Pinho S, and Niehrs C. Dkk3 is required for TGF-β signaling during Xenopus mesoderm induction. Differentiation. 2007;75(10):957-67.
59. Li Y, Liu H, Liang Y, Peng P, Ma X, and Zhang X. DKK3 regulates cell proliferation, apoptosis and collagen synthesis in keloid fibroblasts via TGF-β1/Smad signaling pathway. Biomedicine & Pharmacotherapy. 2017;91:174-80.
60. Lipphardt M, Dihazi H, Jeon N L, Dadafarin S, Ratliff B B, Rowe D W, et al. Dickkopf-3 in aberrant endothelial secretome triggers renal fibroblast activation and endothelial-mesenchymal transition. Nephrology Dialysis Transplantation. 2018;34(1):49-62.
61. Marie J C, Letterio J J, Gavin M, and Rudensky A Y. TGF-β1 maintains suppressor function and Foxp3 expression in CD4+CD25+regulatory T cells. Journal of Experimental Medicine. 2005;201(7):1061-7.
62. Katase N, Lefeuvre M, Gunduz M, Gunduz E, Beder L B, Grenman R, et al. Absence of Dickkopf (Dkk)-3 protein expression is correlated with longer disease-free survival and lower incidence of metastasis in head and neck squamous cell carcinoma. Oncol Lett. 2012;3(2):273-80.
63. Hsieh S-Y, Hsieh P-S, Chiu C-T, and Chen W-Y. Dickkopf-3/REIC functions as a suppressor gene of tumor growth. Oncogene. 2004;23(57):9183-9.
64. Kuphal S, Lodermeyer S, Bataille F, Schuierer M, Hoang B H, and Bosserhoff A K. Expression of Dickkopf genes is strongly reduced in malignant melanoma. Oncogene. 2006;25(36):5027-36.
65. Edamura K, Nasu Y, Takaishi M, Kobayashi T, Abarzua F, Sakaguchi M, et al. Adenovirus-mediated REIC/Dkk-3 gene transfer inhibits tumor growth and metastasis in an orthotopic prostate cancer model. Cancer Gene Therapy. 2007; 14(9):765-72.
66. Yu-Lee L Y, Lee Y C, Pan J, Lin S C, Pan T, Yu G, et al. Bone secreted factors induce cellular quiescence in prostate cancer cells. Sci Rep. 2019;9(1):18635.
67. Veeck J, Bektas N, Hartmann A, Kristiansen G, Heindrichs U, Knüchel R, et al. Wnt signalling in human breast cancer: expression of the putative Wnt inhibitor Dickkopf-3 (DKK3) is frequently suppressed by promoter hypermethylation in mammary tumours. Breast Cancer Research. 2008;10(5):R82.
68. Lorsy E, Topuz A S, Geisler C, Stahl S, Garczyk S, von Stillfried S, et al. Loss of Dickkopf 3 Promotes the Tumorigenesis of Basal Breast Cancer. PloS one. 2016; 11(7):e0160077.
69. Lu K-H, Tounsi A, Shridhar N, Küblbeck G, Klevenz A, Prokosch S, et al. Dickkopf-3 Contributes to the Regulation of Anti-Tumor Immune Responses by Mesenchymal Stem Cells. Frontiers in Immunology. 2015;6.
70. Meister M, Tounsi A, Gaffal E, Bald T, Papatriantafyllou M, Ludwig J, et al. Self-Antigen Presentation by Keratinocytes in the Inflamed Adult Skin Modulates T-Cell Auto-Reactivity. Journal of Investigative Dermatology. 2015;135(8):1996-2004.
71. Chavali M, Klingener M, Kokkosis A G, Garkun Y, Felong S, Maffei A, et al. Non-canonical Wnt signaling regulates neural stem cell quiescence during homeostasis and after demyelination. Nat Commun. 2018;9(1):36.
72. Barrantes IdB, Montero-Pedrazuela A, Guadaño-Ferraz A, Obregon M-J, Mena RMd, Gailus-Durner V, et al. Generation and Characterization of <i>dickkopf3</i>Mutant Mice. Molecular and Cellular Biology. 2006;26(6):2317-26.
73. Hermann M, Pirkebner D, Draxl A, Berger P, Untergasser G, Margreiter R, et al. Dickkopf-3 is expressed in a subset of adult human pancreatic beta cells. Histochemistry and Cell Biology. 2007;127(5):513-21.
74. Pedemonte E, Benvenuto F, Casazza S, Mancardi G, Oksenberg J R, Uccelli A, et al. The molecular signature of therapeutic mesenchymal stem cells exposes the architecture of the hematopoietic stem cell niche synapse. BMC Genomics. 851 2007;8(1):65.
75. Sugimura R, He Xi C, Venkatraman A, Arai F, Box A, Semerad C, et al. Noncanonical Wnt Signaling Maintains Hematopoietic Stem Cells in the Niche. Cell. 2012; 150(2):351-65.
76. Arnold F, Mahaddalkar P U, Kraus J M, Zhong X, Bergmann W, Srinivasan D, et al. Functional Genomic Screening During Somatic Cell Reprogramming Identifies DKK3 as a Roadblock of Organ Regeneration. Advanced Science. 2021;8(14):2100626.
77. Wang X-Q, and Li D-J. The mechanisms by which trophoblast-derived molecules induce maternal-fetal immune tolerance. Cellular & Molecular Immunology. 2020;17(11):1204-7.
78. Aghajanova L, Shen S, Rojas A M, Fisher S J, Irwin J C, and Giudice L C. Comparative Transcriptome Analysis of Human Trophectoderm and Embryonic Stem Cell-Derived Trophoblasts Reveal Key Participants in Early Implantation1. Biology of Reproduction. 2012;86(1).
79. Prater M, Hamilton R S, Wa Yung H, Sharkey A M, Robson P, Abd Hamid N E, et al. RNA-Seq reveals changes in human placental metabolism, transport and endocrinology across the first-second trimester transition. Biology Open. 2021; 10(6).
80. Goldberg J, Pastorello R G, Vallius T, Davis J, Cui Y X, Agudo J, et al. The Immunology of Hormone Receptor Positive Breast Cancer. Frontiers in Immunology. 2021; 12.
81. Abdou Y, Goudarzi A, Yu J X, Upadhaya S, Vincent B, and Carey L A. Immunotherapy in triple negative breast cancer: beyond checkpoint inhibitors. npj Breast Cancer. 2022;8(1):121.
82. Sanjana N E, Shalem O, and Zhang F. Improved vectors and genome-wide libraries for CRISPR screening. Nat Methods. 2014; 11(8):783-4.
83. Hartman Z C, Poage G M, den Hollander P, Tsimelzon A, Hill J, Panupinthu N, et al. Growth of triple-negative breast cancer cells relies upon coordinate autocrine expression of the proinflammatory cytokines IL-6 and IL-8. Cancer Res. 2013;73(11):3470-80.878
84. Reyes A L P, Silva T C, Coetzee S G, Plummer J T, Davis B D, Chen S, et al. GENAVi: a shiny web application for gene expression normalization, analysis and visualization. BMC Genomics. 2019;20(1):745.

### Example 2: Dormant Breast Cancer Tumors Show Susceptibility to Early Anti-DKK3 Antibody Treatment and an Increase in ER

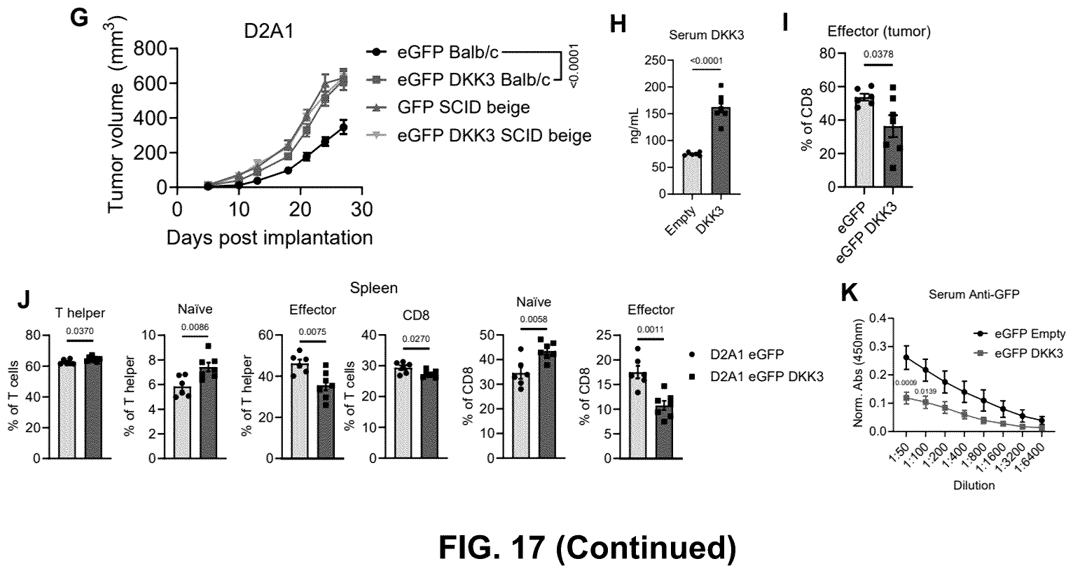

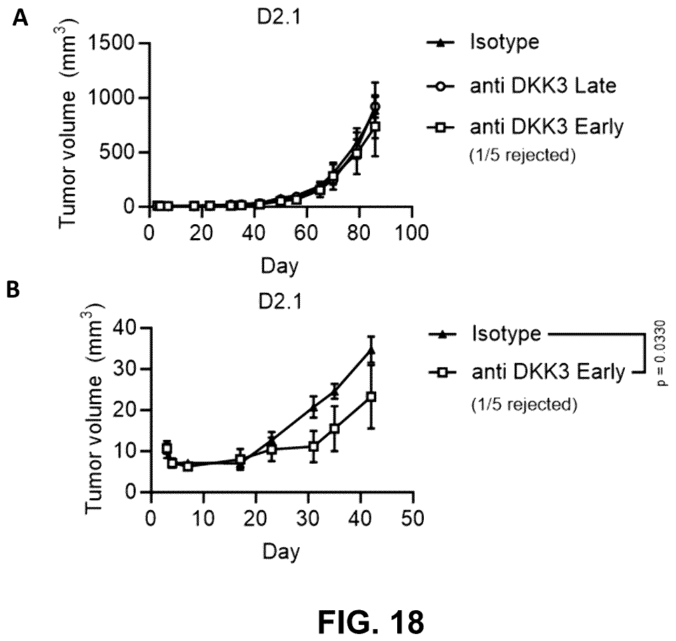

D2.1 cells were implanted into the mammary fat pad of female Balb/c mice to determine the activity of DKK3 blockade in vivo. Beginning on day 3 mice were treated bi-weekly with anti-DKK3 antibodies or isotype control. On day 65, half of the isotype control mice began bi-weekly treatment with anti-DKK3 antibody, creating early (day-3-treated) and late (day-65-treated) cohorts of treated mice. Tumor growth over the duration of the experiment (FIG. 18A) indicates little difference in tumor volume between isotype controls and the anti-DKK3 late cohort. However, tumor growth during the latent phase of the experiment (FIG. 18B) demonstrates early effectiveness of anti-DKK3 blockade (1/5 tumors were rejected completely).

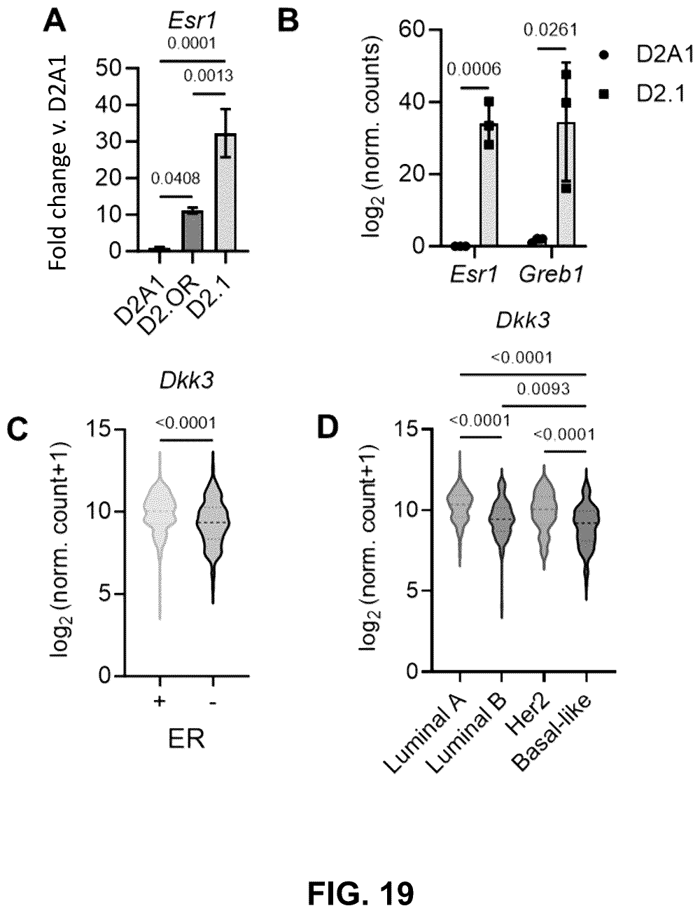

Additionally, qPCR analysis in parental D2A1, D2.OR, and D2.1 cells of Esr1 indicate there is an upregulation of Esr1 in dormant tumors (FIG. 19A). Esr1 and Greb1 gene expression from RNAseq of D2.1 or D2A1 cells is also analyzed (FIG. 19B). While there seems to be differential regulation of Esr1 in dormant tumors, there does not seem to be a difference in Dkk3 expression in ER-positive vs ER-negative BCs (FIG. 19C). However, other molecular subtypes of BC—such as Luminal A, Luminal B, Her2, and Basal-like subtypes—possess differential expression of Dkk3 (FIG. 19D).

We claim:

1. A method of inhibiting growth of a dormant cancer cell comprising administering an effective amount of a DKK3 inhibitor to a subject in need thereof to inhibit growth of the dormant cancer cell.

2. The method of claim 1, wherein the dormant cancer cell is a cell from a cancer selected from the group consisting of a breast cancer, a pancreatic cancer, a prostate cancer, a brain cancer, an ovarian cancer, a liver cancer, and a lung cancer.

3. The method of claim 2, wherein the dormant cancer cell is from a triple-negative breast cancer or an estrogen receptor positive breast cancer.

4. The method of claim 1, wherein the administration results in an increase in Foxp3+T regulatory cells in the subject.

5. The method of claim 1, wherein the subject previously received a cancer therapy treatment for a cancer.

6. The method of claim 5, wherein the cancer therapy treatment for the cancer resulted in remission of the cancer.

7. The method of claim 1, wherein the DKK3 inhibitor is an antibody or antibody fragment.

8. The method of claim 1, wherein the administration of the effective amount of the DKK3 inhibitor increases CD8+T cell proliferation and/or decreases CD4+T cell proliferation.

9. The method of claim 1, wherein the dormant cancer cell comprises at least one metastasis.

10. The method of claim 1, wherein the dormant cancer cell is part of at least one secondary tumor.

11. The method of claim 1, further comprising administering an immunotherapy.

12. The method of claim 11, wherein the immunotherapy comprises an immune checkpoint inhibitor.

13. The method of claim 12, wherein the immune checkpoint inhibitor is selected from the group consisting of a CTLA-4 antagonist, a PD-1 antagonist, a PD-L1 antagonist, an OX40 agonist, a LAG3 antagonist, a 4-1BB agonist, or a TIM3 antagonist.

14. The method of claim 1, further comprising administering at least one additional cancer therapy.

15. The method of claim 14, wherein the at least one additional cancer therapy is selected from the group consisting of an anti-cancer vaccine, radiation, chemotherapy, surgery, immunotherapy, gene therapy, hormone therapy, anti-angiogenic therapy, and cytokine therapy.

16. A method of inhibiting CD4+Treg cells in a subject comprising administering an effective amount of a DKK3 inhibitor to a subject in need of inhibition of a CD4+T reg cell response, wherein subject has a decrease in Foxp3+T regulatory cells after administration of the DKK3 inhibitor.

17. The method of claim 16, wherein the DKK3 inhibitor is an antibody or antibody fragment.

18. The method of claim 16, wherein the administration of the effective amount of the DKK3 inhibitor increases CD8+T cell proliferation and/or decreases CD4+T cell proliferation.

19. A method of inhibiting cancer recurrence in a subject previously treated for a cancer comprising administering an effective amount of a DKK3 inhibitor to the subject following completion of a cancer therapy in the subject.

20. A method of treating a subject with cancer comprising: (a) obtaining a sample of cancer cells from the subject; (b) determining the level of expression of DKK3 in the cancer cells; (c) treating the cancer in the subject; (d) administering a DKK3 inhibitor to the subject with increased expression levels of DKK3 in the cancer cells in step (b) after completion of treatment of the subject for the cancer in step (c).


SEQUENCE LISTING (1 sequence)

SEQ ID NO: 1 - DNA, 21 bp, synthetic construct
  source 1..21: mol_type = other DNA
tctgtgacaaccagagggatt